(19) United States
(12) Patent Application Publication
(10) Pub. No.: US 2022/0153863 A1
(43) Pub. Date: May 19, 2022

(54) PRAME BINDING MOLECULES AND USES THEREOF

(71) Applicants: Hans David Staffan Ulmert, Santa Monica, CA (US); Richard John O'Reilly, Roxbury, CT (US); Ivo Lorenz, New York, NY (US); Mary Ann Pohl, New York, NY (US); Namita Trikannad, Oakland, CA (US)

(72) Inventors: Hans David Staffan Ulmert, Santa Monica, CA (US); Richard John O'Reilly, Roxbury, CT (US); Ivo Lorenz, New York, NY (US); Mary Ann Pohl, New York, NY (US); Namita Trikannad, Oakland, CA (US)

(21) Appl. No.: 17/438,653

(22) PCT Filed: Mar. 13, 2020
(86) PCT No.: PCT/US20/22729

§ 371 Date: Sep. 13, 2021

Related U.S. Application Data
(60) Provisional application No. 62/817,999, filed on Mar. 13, 2019.

(51) Int. Cl. C07K 16/30; A61K 47/68; A61K 51/10; G01N 33/574

(52) U.S. Cl. CPC C07K 16/30; A61K 47/6803; C07K 2317/92; A61K 51/1045; G01N 33/57492; A61K 47/6851

(57) ABSTRACT

The present invention provides various PRAME binding molecules (including antibodies, antibody fragments, chimeric antigen receptors, and the like), compositions and cells (including T cells) comprising such PRAME binding molecules, and methods of using such PRAME binding molecules, compositions and cells, for example in the detection and/or monitoring of PRAME-positive tumors.

### CROSS-REFERENCE TO RELATED APPLICATIONS

This application claims the benefit of priority of U.S. Provisional Patent Application No. 62/817,999 filed on Mar. 13, 2019, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 13, 2020, is named MSKCC_041_WO1_SL.txt and is 29,864 bytes in size.

### INCORPORATION BY REFERENCE

For the purposes of only those jurisdictions that permit incorporation by reference, all of the references cited in this disclosure are hereby incorporated by reference in their entireties (numbers in parentheses or in superscript following text in this patent disclosure refer to the numbered references provided in the “Reference List” in the Appendix section of this patent specification). In addition, any manufacturers' instructions or catalogues for any products cited or mentioned herein are incorporated by reference. Documents incorporated by reference into this text, or any teachings therein, can be used in the practice of the present invention.

### BACKGROUND

There is an urgent need for immuno-theranostics that could be applied to a broader spectrum of malignancies. Recent breakthroughs in immunobiotechnology have led to new and potent therapeutics for a number of both malignant and non-malignant diseases. In parallel, important discoveries in radiochemistry and PET-technology have made it possible to image the biodistribution of cancer specific antibodies and the arming of such antibodies with extremely cytotoxic alpha-emitting radiometals. However, all of these advances rely on the ability of the antibody to specifically target the malignant cells. Currently, most of the successful antibodies and immunoconjugates are used to treat only a limited number of cancers such as HER2+ breast cancers.

Preferentially Expressed Antigen in Melanoma (PRAME) is a cancer/testis antigen initially isolated from melanoma cells. PRAME has been shown to be overexpressed in an array of solid and hematological malignancies. In hematological malignancies, PRAME has displayed particularly high expression in Acute Myeloid Leukemia (AML), Acute Lymphoid Leukemia (ALL) and Chronic Myeloid Leukemia (CIVIL) in blast crisis (1, 2). Among the prevalent solid tumors, PRAME is particularly highly expressed in ovarian carcinomas, endometrial carcinomas, squamous cell carcinomas of the lung, cutaneous melanomas and basal subtype breast cancer (3). In addition, PRAME is expressed in several pediatric cancers, such as medulloblastoma, neuroblastoma and osteosarcoma. Although the pathophysiological function of PRAME remains unknown, data indicates that PRAME could be a repressor of the Retinoic Acid Receptor (RAR) and thereby antagonize the antiproliferative and cytotoxic effects of Retinoic Acid (RA) (4). PRAME also has predictive capacity as a disease biomarker. For example, in solid tumors, PRAME expression has been associated with very poor prognosis (5). In CIVIL, PRAME expression is higher in the blast phase, suggesting a role in disease progression (6).

Although PRAME is commonly viewed as an intracellular protein, studies have now shown that PRAME can be localized in the plasma membrane (7, 8). Furthermore, in vivo efficacy of radiolabeled antibodies targeting the exposed plasma membrane domain of PRAME has now also been demonstrated (8). The expression profile of PRAME in a wide array of both liquid and solid cancers, coupled with recent immuno-technological advances permitting the generation of high affinity humanized monoclonal antibodies (mAbs) raises the prospect that new PRAME-targeted drugs could potentially become an important addition to the immunotherapeutic armamentarium.

### SUMMARY OF THE INVENTION

The present invention provides novel PRAME-binding molecules (including, but not limited to, antibodies, antibody fragments, and molecules comprising antibodies or antibody fragments) that bind to an extracellular domain of PRAME, and compositions and cells comprising such PRAME-binding molecules. The present invention also provides various methods of use of such PRAME binding molecules, for example in the detection and monitoring (e.g. “theranostics”) of an array of PRAME expressing cancers across a spectrum of cancer stages.

As described further in the Examples section of this patent specification a portion of the PRAME protein (UniProt accession number P78395) corresponding to amino acids Arg310-Asn331, which are predicted to be exposed on the extracellular side of the plasma membrane, was synthesized as a peptide conjugated to either biotin or bovine serum albumin (BSA) for in vitro antibody generation. A proprietary naïve, semi-synthetic scFv phage display library was screened for antibodies that bind the PRAME peptide using standard solution phage display panning techniques. PRAME peptide conjugated to biotin was incubated with the phage library and captured with paramagnetic streptavidin beads, followed by standard washing, elution and phage amplification steps. Prior to incubating the phage library with PRAME peptide, the library was depleted of non-specific binding phage by incubation with a PRAME family consensus sequence peptide conjugated to biotin to remove all phage displaying antibodies that bind PRAME homologs. The entire process of panning was repeated 3 times, using amplified PRAME target binder-enriched phage pools from the previous round of panning as input for subsequent rounds. Six unique antibodies (B029_1A6, B029_1A7, B029_1G7, B029_1H1, B029_2D4, and B029_2H1) that showed specific binding to the PRAME peptide and to PRAME+ cells were identified. These six antibodies, as well as a variety of other PRAME binding molecules containing binding determinants (e.g. complementarity determining regions or CDRs) and/or variable domains derived from those present in these six novel anti-PRAME antibodies, are further described herein. Uses of such PRAME binding molecules are also described herein.

Accordingly, in certain embodiments the present invention provides the anti-PRAME antibodies referred to herein as B029_1A6, B029_1A7, B029_1G7, B029_1H1, B029_2D4, and B029_2H1, as well as various PRAME binding molecules related to, derived from, or containing, these antibodies or portions of these antibodies (such as the CDRs and/or variable domains of these antibodies). The amino acid sequences of each of these six antibodies, and all other sequences referred to herein using a sequence identification number (i.e. a “SEQ ID NO.”), are provided in Tables 1 through 6 of the “Detailed Description” section of this patent disclosure. Table 7 provides a summary of the various sequence identification numbers (i.e. SEQ ID No.$). The amino acid sequences of these six antibodies are also provided in the Appendix 2 to this patent application.

In one embodiment, the present invention provides an isolated PRAME binding molecule comprising a light chain variable region comprising: a CDR L1 domain comprising SEQ ID NO. 5, a CDR L2 domain comprising SEQ ID NO. 6, and a CDR L3 domain comprising SEQ ID NO. 7, and a heavy chain variable region comprising: a CDR H1 domain comprising SEQ ID NO. 8, a CDR H2 domain comprising SEQ ID NO. 9, and a CDR H3 domain comprising SEQ ID NO. 10, and—i.e. the CDRs of B029_1A6.

In another embodiment, the present invention provides an isolated PRAME binding molecule comprising a light chain variable region comprising: a CDR L1 domain comprising SEQ ID NO. 15, a CDR L2 domain comprising SEQ ID NO. 16, and a CDR L3 domain comprising SEQ ID NO. 17, and a heavy chain variable region comprising: a CDR H1 domain comprising SEQ ID NO. 18, a CDR H2 domain comprising SEQ ID NO. 19, and a CDR H3 domain comprising SEQ ID NO. 20—i.e. the CDRs of B209_1A7.

In another embodiment, the present invention provides an isolated PRAME binding molecule comprising a light chain variable region comprising: a CDR L1 domain comprising SEQ ID NO. 25, a CDR L2 domain comprising SEQ ID NO. 26, and a CDR L3 domain comprising SEQ ID NO. 27, and a heavy chain variable region comprising: a CDR H1 domain comprising SEQ ID NO. 28, a CDR H2 domain comprising SEQ ID NO. 29, and a CDR H3 domain comprising SEQ ID NO. 30—i.e. the CDRs of B209_1G7.

In another embodiment, the present invention provides an isolated PRAME binding molecule comprising a heavy chain variable region comprising: a light chain variable region comprising: a CDR L1 domain comprising SEQ ID NO. 35, a CDR L2 domain comprising SEQ ID NO. 36, and a CDR L3 domain comprising SEQ ID NO. 37, and a CDR H1 domain comprising SEQ ID NO. 38, a CDR H2 domain comprising SEQ ID NO. 39, and a CDR H3 domain comprising SEQ ID NO. 40—i.e. the CDRs of B209_1H1

In another embodiment, the present invention provides an isolated PRAME binding molecule comprising a light chain variable region comprising: a CDR L1 domain comprising SEQ ID NO. 45, a CDR L2 domain comprising SEQ ID NO. 46, and a CDR L3 domain comprising SEQ ID NO. 47, and a heavy chain variable region comprising: a CDR H1 domain comprising SEQ ID NO. 48, a CDR H2 domain comprising SEQ ID NO. 49, and a CDR H3 domain comprising SEQ ID NO. 50—i.e. the CDRs of B209_2D4.

In another embodiment, the present invention provides an isolated PRAME binding molecule comprising a light chain variable region comprising: a CDR L1 domain comprising SEQ ID NO. 55, a CDR L2 domain comprising SEQ ID NO. 56, and a CDR L3 domain comprising SEQ ID NO. 57, and a heavy chain variable region comprising: a CDR H1 domain comprising SEQ ID NO. 58, a CDR H2 domain comprising SEQ ID NO. 59, and a CDR H3 domain comprising SEQ ID NO. 60—i.e. the CDRs of B209_2H1.

In another embodiment, the present invention provides an isolated PRAME binding molecule comprising a light chain variable region comprising SEQ ID NO. 1, and a heavy chain variable region comprising SEQ ID NO.3—i.e. the variable regions of B029_1A6.

In another embodiment, the present invention provides an isolated PRAME binding molecule comprising a light chain variable region comprising SEQ ID NO. 11, and a heavy chain variable region comprising SEQ ID NO.13—i.e. the variable regions of B029_1A7.

In another embodiment, the present invention provides an isolated PRAME binding molecule comprising a light chain variable region comprising SEQ ID NO. 21, and a heavy chain variable region comprising SEQ ID NO.23—i.e. the variable regions of B029_1G7.

In another embodiment, the present invention provides an isolated PRAME binding molecule comprising a light chain variable region comprising SEQ ID NO. 31, and a heavy chain variable region comprising SEQ ID NO.33—i.e. the variable regions of B029_1H1.

In another embodiment, the present invention provides an isolated PRAME binding molecule comprising a light chain variable region comprising SEQ ID NO. 41, and a heavy chain variable region comprising SEQ ID NO.43—i.e. the variable regions of B029_2D4.

In another embodiment, the present invention provides an isolated PRAME binding molecule comprising a light chain variable region comprising SEQ ID NO. 51, and a heavy chain variable region comprising SEQ ID NO.53—i.e. the variable regions of B029_2H1.

In other embodiments, the present invention also provides isolated PRAME binding molecules that are able to specifically bind to the same epitope on PRAME as any one of the PRAME binding molecules described above. Similarly, in some embodiments the present invention provides isolated PRAME binding molecules that are able to compete with any one of the PRAME binding molecules described above for binding to PRAME.

In some embodiments, the PRAME binding molecules of the invention (such as those described above) are antibodies. For example, in some embodiments a PRAME binding molecule of the invention may be a humanized antibody, a fully human antibody, a murine antibody, a chimeric antibody, a monoclonal antibody, a polyclonal antibody, a bi-specific antibody, or a multi-specific antibody.

In some embodiments, the PRAME binding molecules of the invention (such as those described above) are, or comprise, antibody fragments. For example, in some embodiments a PRAME binding molecule of the invention may be, or may comprise, a Fv, a Fab, a F(ab′)2, a Fab′, a dsFv fragment, a single chain Fv (scFV), an sc(Fv)2, a disulfide-linked (dsFv), a diabody, a triabody, a tetrabody, a minibody, a single chain antibody.

In some embodiments, the PRAME binding molecules of the invention (such as those described above) are, or comprise, or are comprised within, chimeric antigen receptors (CARs). For example, in some embodiments the present invention provides chimeric antigen receptors (CARs) that comprise a PRAME binding molecule of the invention. Such CARs will typically comprise an scFV that is a PRAME binding molecule as described herein.

In some embodiments, the PRAME binding molecules of the invention (such as those described above) are, or comprise, or are comprised within, bi-specific T cell engagers (biTEs”). Such BiTEs will typically comprise two scFVs, one of which is a PRAME binding molecule as described herein.

In some embodiments, the PRAME binding molecules of the invention (such as those described above) are, or comprise, or are comprised within, radio-immuno therapeutic (RIT) compounds.

In some embodiments, the PRAME binding molecules of the invention (such as those described above) are, or comprise, or are comprised within, radio-diagnostic PET-compounds.

In some embodiments, the PRAME binding molecules of the invention are conjugated to therapeutic agents and/or imaging agents. In this way, the PRAME binding molecules can be used to target therapeutic agents or imaging agents to a PRAME-expressing tumor cells or tumors. For example, in some embodiments, the PRAME binding molecules can be conjugated to cytotoxic agents, such chemotherapeutic agents or radionuclides (such as the alpha-emitting radionuclide Actinium-225). In other embodiments the PRAME binding molecules can be conjugated to imaging agents, such as a positron-emitting radiolabel useful for PET imaging (such as zirconium-89).

In those embodiments where a PRAME binding molecule of the invention comprises a heavy chain constant region, or a portion thereof, the heavy-chain constant region may be an alpha, delta, epsilon, gamma, or mu heavy chain constant region. Similarly, in some embodiments, the PRAME binding molecules of the invention (such as those described above) may be, or may comprise, an IgA, IgD, IgE, IgG or IgM class immunoglobulin molecule.

In those embodiments where a PRAME binding molecule of the invention comprises a light chain constant region, or a portion thereof, the light-chain constant region may a lambda light chain constant region or a kappa light chain constant region.

In addition to the various PRAME binding molecules described above, in some embodiments the present invention also provides compositions comprising such PRAME binding molecules, for example pharmaceutical compositions which comprise a PRAME binding molecule and a pharmaceutically acceptable carrier.

In further embodiments the present invention also provides cells that produce a PRAME binding molecule as described herein, such as mammalian cells (including human and murine cells). For example, in some embodiments the present invention provides T cells that express a chimeric antigen receptor—i.e. CAR T cells—wherein the chimeric antigen receptor expressed by the CAR T cells is and/or comprises a PRAME binding molecule as described herein. For example, such a CAR T cells may comprise a CAR that comprises an scFV that is a PRAME binding molecule as described herein.

In yet further embodiments the present invention also provides nucleotide sequences that encode the PRAME binding molecules described herein, as well as vectors and host cells (including human and murine host cells, such as T cells) comprising such nucleotide sequences.

The present invention also provides various different methods of use of the PRAME binding molecules, compositions, and cells described herein.

For example, in some embodiments the present invention provides methods for inhibiting the proliferation of, and/or killing, tumor cells. Such methods involve contacting tumor cells with an effective amount of a PRAME binding molecule or composition (such as pharmaceutical composition) or cells (such as CAR T cells), as described herein. In other embodiments, the present invention provides methods for inhibiting a PRAME biological activity in cells or in a tissue. Such methods involve delivering an effective amount of a PRAME binding molecule or composition (such as pharmaceutical composition) or cells (such as CAR T cells) to cells or a tissue that expresses or contains PRAME. For example, in some embodiments, PRAME binding molecules of the invention are used to kill tumor cells by antibody-dependent cell-mediated cytotoxicity (ADCC). In some embodiments, PRAME binding molecules of the invention are used to kill tumor cells by conjugation of the PRAME binding molecule to a cytotoxic agent. In some embodiments PRAME binding molecules of the invention are used to kill tumor cells using a CAR T cell having a CAR that comprises a PRAME binding molecule.

In each of the above methods the tumor cells may be any PRAME-positive tumor cells.

The present invention also provides methods for detecting PRAME-expressing tumor cells in a sample (such as in a cell or tissue sample—e.g. a biopsy sample) or in a living subject. Such methods typically involve contacting the PRAME-expressing tumor cells with a PRAME binding molecule as described herein, and detecting binding of the PRAME binding molecule to PRAME. In some embodiments the PRAME binding molecule is conjugated to an imaging agent. In some such embodiments the imaging agent is a positron-emitting agent and binding of the PRAME molecule to PRAME is detected by positron emission tomography (PET) imaging.

The present invention also provides methods for determining whether a PRAME binding molecule inhibits biological activity of PRAME in a cell or tissue sample, by contacting the cell or tissue sample with a PRAME binding molecule and assessing a biological activity of PRAME.

In each of the above methods the tumor cells may be any PRAME-positive tumor cells. In some embodiments the tumor cells may be, or may be from, acute myeloid leukemia (AML), acute lymphoid leukemia (ALL), chronic myeloid leukemia (CIVIL), ovarian carcinoma, endometrial carcinoma, lung carcinoma (e.g. squamous cell carcinomas of the lung), melanoma, (e.g. cutaneous melanoma), breast cancer (e.g. basal subtype breast cancer), medulloblastoma, neuroblastoma, or osteosarcoma tumor cells. In some of such methods the tumor cells overexpress, or exhibit over-activity of, PRAME. In some of such methods the tumor cells are in vitro, while in other methods the tumor cells are in vivo.

These and other embodiments of the invention are further described in the “Brief Description of the Figures,” “Detailed Description,” “Examples,” “Figures,” and “Claims” sections of this patent disclosure, each of which sections is intended to be read in conjunction with, and in the context of, all other sections of the present patent disclosure. Furthermore, one of skill in the art will recognize that the various embodiments of the present invention described herein can be combined in various different ways, and that such combinations are within the scope of the present invention.

### BRIEF DESCRIPTION OF THE FIGURES

FIG. 1 shows flow cytometry analyses of mAb binding to 6 cell lines: U937, PRAME+ human myeloid leukemia; HL60, PRAME+ human acute promyelocytic leukemia; Molm-13 PRAME+ human acute monocytic leukemia; BLCL, PRAME− Epstein-Barr virus-transformed lymphoblastoid B cells; and NK92, PRAME− immortalized natural killer-like cells. The individual bars shown for each of the indicated mAbs/concentrations are data for, from left-to right, U937, HL60, Molm-13, BLCL and NK92 cells. Data for a secondary antibody only control, a rabbit polyclonal antibody (RpAb), and a rabbit secondary antibody control are also shown.

FIG. 2 shows an estimation of antibody affinity to THP-1 cells determined by flow cytometry with titrated concentrations of antibody, and EC50 values for binding. MPA1 (Pankov et al., 2017), a rabbit anti-PRAME polyclonal antibody was included as reference for comparison.

### DETAILED DESCRIPTION

Some of the main embodiments of the present invention are described in the “Summary of the Invention,” “Examples,” “Brief Description of the Figures,” and “Figures” sections of this patent disclosure. This Detailed Description section provides certain additional description and details and is intended to be read in conjunction with all other sections of the present patent disclosure.

The present invention provides molecules that bind to PRAME—referred to herein as “PRAME binding molecules”. Such PRAME binding molecules are antibodies, or antigen-binding fragments thereof, are molecules that comprise such antibodies, or antigen-binding fragments thereof, and which specifically bind to PRAME.

Polynucleotides that encode the PRAME binding molecules described herein, as well as compositions comprising the PRAME binding molecules, and methods of making the PRAME binding molecules, are also provided.

Methods of using the novel PRAME binding molecules described herein are also provided, such as methods of treating cancer and/or inhibiting proliferation of cancer cells, and methods of diagnosing or monitoring cancer.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Ausubel et al. eds. (2015) Current Protocols in Molecular Biology (John Wiley and Sons); Greenfield, ed. (2013) Antibodies: A Laboratory Manual (2nd ed., Cold Spring Harbor Press); Green and Sambrook, eds. (2012), Molecular Cloning: A Laboratory Manual (4th ed., Cold Spring Harbor Laboratory Press); Krebs et al., eds. (2012) Lewin's Genes XI (11th ed., Jones & Bartlett Learning); Freshney (2010) Culture Of Animal Cells (6th ed., Wiley); Weir and Blackwell, eds., (1996) Handbook Of Experimental Immunology, Volumes I-IV (5th ed., Wiley-Blackwell); Borrebaeck, ed. (1995) Antibody Engineering (2nd ed., Oxford Univ. Press); Glover and Hames, eds., (1995) DNA Cloning: A Practical Approach, Volumes I and II (2nd ed., IRL Press); Rees et al., eds. (1993) Protein Engineering: A Practical Approach (1st ed., IRL Press); Mayer and Walker, eds. (1987) Immunochemical Methods In Cell And Molecular Biology (Academic Press, London); Nisonoff (1984) Introduction to Molecular Immunology (2nd ed., Sinauer Associates, Inc.); and Steward (1984) Antibodies: Their Structure and Function (1st ed., Springer Netherlands).

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

### I. Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values±10% of the stated number.

Numbers in parentheses or superscript following text in this patent disclosure refer to the numbered references provided in the “Reference List” section at the end of this patent disclosure.

Wherever embodiments are described with the language “comprising,” otherwise analogous embodiments described in terms of “consisting of” and/or “consisting essentially of” are included.

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin” are used interchangeably herein.

A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region (CL). The light chain constant region is comprised of one domain, Cl. The variable regions of the heavy and light chains contain a binding domain that interacts with an antigen. The constant regions of the antibodies can mediate the binding of the immunoglobulin to host tissues or factors, including various cells of the immune system (e.g. effector cells) and the first component (C1q) of the classical complement system.

Antibodies can be of any the five major classes of immunoglobulins: IgA, IgD, IgE, IgG and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu respectively. The different classes of immunoglobulins have different and well-known subunit structures and three-dimensional configurations. There are two classes of mammalian light chains, lambda and kappa. I

The VH and VL regions can be further subdivided into regions of hypervariability, termed complementarity-determining regions (CDRs), interspersed with regions that are more conserved, termed framework (FW) regions. The CDRs in each chain are held together in close proximity by the FW regions and, with the CDRs from the other chain, contribute to the formation of the antigen-binding site of antibodies. Each VH and VL is composed of three CDRs and four FW regions, arranged from amino-terminus to carboxy-terminus in the following order: FW1, CDR1, FW2, CDR2, FW3, CDR3, FW4.

There are at least two techniques for determining CDRs: (1) an approach based on cross-species sequence variability (Kabat et al., Sequences of Proteins of Immunological Interest, 5th Ed. Public Health Service, National Institutes of Health, Bethesda, Md. (1991)); and (2) an approach based on crystallographic studies of antigen-antibody complexes (Al-lazikani et al., J. Molec. Biol. 273:927-948 (1997)). In addition, combinations of these two approaches are sometimes used in the art to determine CDRs.

The amino acid position numbering as in Kabat, refers to the numbering system used for heavy chain variable domains or light chain variable domains (approximately residues 1-107 of the light chain and residues 1-113 of the heavy chain). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a, according to Kabat) after residue 52 of H2 and inserted residues (e.g., residues 82a, 82b, and 82c, etc., according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. See Table 1.

IMGT (ImMunoGeneTics) also provides a numbering system for the immunoglobulin variable regions, including the CDRs. See, e.g., Lefranc, M. P. et al., Dev. Comp. Immunol. 27: 55-77 (2003). The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used herein, the term “antibody” encompasses polyclonal antibodies; monoclonal antibodies; multispecific antibodies, such as bispecific antibodies generated from at least two intact antibodies; humanized antibodies; human antibodies; chimeric antibodies; fusion proteins comprising an antigen-determination portion of an antibody; and any other modified immunoglobulin molecule comprising an antigen recognition site, so long as the antibodies exhibit the desired biological activity.

A “monoclonal antibody” (mAb) refers to a homogeneous antibody population that is involved in the highly specific recognition and binding of a single antigenic determinant, or epitope. This is in contrast to polyclonal antibodies, which typically include different antibodies directed against different antigenic determinants. The term “monoclonal” can apply to both intact and full-length monoclonal antibodies, as well as to antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 and 5,639,641.

The term “human antibody” means an antibody produced by a human or an antibody having an amino acid sequence corresponding to an antibody produced by a human made using any technique known in the art. The definition of a human antibody includes intact or full-length antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “antigen-binding fragment” refers to a portion of an intact antibody comprising the complementarity determining variable regions of the antibody. Examples of antibody fragments that can constitute an “antigen-binding fragment” include, but are not limited to, Fab, Fab′, F(ab′)2, and Fv fragments, linear antibodies, single chain antibodies (e.g., ScFvs), and multi-specific antibodies formed from antibody fragments.

A “blocking” antibody or an “antagonist” antibody is one that inhibits or reduces biological activity of the antigen it binds, such as PRAME. In certain aspects, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “germlining” means that amino acids at specific positions in an antibody are mutated back to those in the germ line.

The “IgG1 triple mutant” or “IgG1-TM” antibody format is a human IgG1 isotype containing three single amino acid substitutions, L234F/L235E/P331S, within the lower hinge and CH2 domain (Oganesyan et al., Acta Crystallogr. D Biol. Crystallogr. 64:700-704, 2008). The TM causes a profound decrease in binding to human FcγRI, FcγRII, FcγRIII, and C1q, resulting in a human isotype with very low effector function.

The terms “YTE” or “YTE mutant” or “YTE mutation” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Immunol. 169:5171-5180 (2002); Dall'Acqua et al., J. Biol. Chem. 281:23514-24 (2006); Robbie et al., Antimicrob. Agents Chemother. 57, 6147-6153 (2013)). See also U.S. Pat. No. 7,083,784, which is hereby incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer.

The affinity or avidity of an antibody for an antigen can be determined experimentally using any suitable method known in the art, e.g., flow cytometry, enzyme-linked immunosorbent assay (ELISA), or radioimmunoassay (MA), or kinetics (e.g., KINEXA® or BIACORE™ or OCTET® analysis). Direct binding assays as well as competitive binding assay formats can be readily employed. (See, e.g., Berzofsky et al., “Antibody-Antigen Interactions,” In Fundamental Immunology, Paul, W. E., ed., Raven Press: New York, N.Y. (1984); Kuby, Immunology, W. H. Freeman and Company: New York, N.Y. (1992)). The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art.

“Potency” is normally expressed as an IC₅₀ (or EC₅₀) value, in nM or pM, unless otherwise stated. IC₅₀ is the median inhibitory concentration of an antibody molecule. In functional assays, IC₅₀ is the concentration that reduces a biological response by 50% of its maximum. In ligand-binding studies, IC₅₀ is the concentration that reduces receptor binding by 50% of maximal specific binding level. IC₅₀ can be calculated by any number of means known in the art.

The fold improvement in potency for the antibodies or polypeptides of the invention as compared to a reference antibody can be at least about 2-fold, at least about 4-fold, at least about 6-fold, at least about 8-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more.

The terms “inhibit,” “block,” and “suppress” are used interchangeably and refer to any statistically significant decrease in a given biological activity, including full blocking of the activity. For example, “inhibition” can refer to a decrease of about 10%, 20%, 30%, 40%, 50%, 60%, 70%, 80%, 90% or 100% in biological activity. Accordingly, when the terms “inhibition” or “suppression” are applied to describe, e.g., an effect of a PRAME binding molecule, the terms may refer to the ability of an PRAME binding molecule to statistically significantly decrease the proliferation of or survival of a PRAME-expressing tumor cell, and the like. Inhibition may be determined relative to an untreated control—for example a control not treated with the PRAME binding molecule. In some embodiments, a PRAME binding molecule can inhibit an activity by at least 10%, or at least 20%, or at least 30%, or at least 40%, or at least 50%, or at least 60%, or at least 70%, or at least 80%, or at least 90% or about 100%, as determined, for example, by flow cytometry, Western blotting, ELISA, proliferation assays, or other assays known to those of skill in the art.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g. acetate, phosphate or citrate buffer), a surfactant (e.g. polysorbate), a stabilizing agent (e.g. human albumin), a preservative (e.g. benzyl alcohol), an absorption promoter to enhance bioavailability and/or other conventional solubilizing or dispersing agents.

An “effective amount” of a binding molecule as disclosed herein is an amount sufficient to carry out a specifically stated purpose. An “effective amount” can be determined empirically and in a routine manner, in relation to the stated purpose.

The PRAME binding molecules of the invention can be naked or conjugated to other molecules such as toxins, labels, etc. The term “label” when used herein refers to a detectable compound or composition that is conjugated directly or indirectly to a binding molecule, so as to generate a “labeled” binding molecule. The label can be detectable by itself (e.g., radioisotope labels or fluorescent labels) or, as in the case of, e.g., an enzymatic label, can catalyze chemical alteration of a substrate compound or composition that is detectable.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

“Prevent” or “prevention” refer to prophylactic or preventative measures that prevent and/or slow the development or recurrence of a targeted pathologic condition or disorder. Thus, those in need of prevention include those prone to have or susceptible to the disorder, including those who have had the disorder and are susceptible to recurrence. In certain embodiments, a disease or disorder is successfully prevented according to the methods provided herein if the patient develops, transiently or permanently, e.g., fewer or less severe symptoms or pathology associated with the disease or disorder, or a later onset of symptoms or pathology associated with the disease or disorder, than a patient who has not been subject to the methods of the invention. In some embodiments, recurrence of cancer is prevented for at least about 3, 6, 9, 12, 18, or 24 months after the start of treatment with a PRAME binding molecule of the invention.

The terms “polypeptide,” “peptide,” and “protein” are used interchangeably herein to refer to polymers of amino acids of any length. The polymer can be linear or branched, it can comprise modified amino acids and non-amino acids can interrupt it. The terms also encompass an amino acid polymer that has been modified naturally or by intervention; for example, disulfide bond formation, glycosylation, lipidation, acetylation, phosphorylation or any other manipulation or modification such as conjugation with a labeling component. Also included within the definition are, for example, polypeptides containing one or more analogs of an amino acid (including, for example, unnatural amino acids, etc.), as well as other modifications known in the art. In certain embodiments, the polypeptides can occur as single chains or associated chains.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the amino acid sequences of the binding molecules of the invention do not abrogate the binding of the binding molecule to the antigen(s), i.e., PRAME to which the binding molecule binds. Methods of identifying conservative nucleotide and amino acid substitutions which do not eliminate antigen-binding are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. U.S.A. 94:412-417 (1997)).

A “polynucleotide,” as used herein can include one or more “nucleic acids,” “nucleic acid molecules,” or “nucleic acid sequences,” and refers to a polymer of nucleotides of any length, and includes DNA and RNA. The polynucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

The term “vector” means a construct, which is capable of delivering and, in some embodiments expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

An “isolated” polypeptide, antibody, binding molecule, polynucleotide, vector, or cell is in a form not found in nature. Isolated polypeptides, antibodies, binding molecules, polynucleotides, vectors, or cells include those which have been purified to a degree that they are no longer in a form in which they are found in nature. In some embodiments, a polypeptide, antibody, binding molecule, polynucleotide, vector, or cell that is isolated is substantially pure. When used herein, the term “substantially pure” refers to purity of greater than 75%, preferably greater than 80% or 90%, and most preferably greater than 95%.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)), can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain embodiments, the default parameters of the alignment software are used.

In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Other terms are defined elsewhere in this patent disclosure, or else are used in accordance with their usual meaning in the art.

### II. PRAME Binding Molecules

The acronym “PRAME” refers to “Preferentially Expressed Antigen in Melanoma.” The PRAME protein, and the nucleotide sequences that encode it are well known in the art. For example, PRAME nucleotide and amino acid sequences are publicly available, for example in the GenBank/NCBI database.

The terms “PRAME binding molecule” or “binding molecule that binds to PRAME” or “anti-PRAME” refer to a binding molecule that is capable of binding PRAME with sufficient affinity such that the binding molecule is useful for one of the applications described herein. Typically, a binding molecule that “specifically binds” to PRAME binds to an unrelated, non-PRAME protein to an extent of less than about 10% of the binding of the binding molecule to PRAME, as measured, e.g., by a radioimmunoassay (RIA), BIACORE™ (e.g. using recombinant PRAME as the analyte and binding molecule as the ligand, or vice versa), KINEXA®, OCTET®, or other binding assays known in the art. In certain embodiments, binding molecule that binds to PRAME has a dissociation constant (K_{D}) of ≤1 μM, ≤100 nM, ≤10 nM, ≤1 nM, ≤0.1 nM, ≤10 pM, ≤1 pM, or ≤0.1 pM.

Exemplary PRAME binding molecules of the present invention include the six “lead” antibody clones referred to herein as B029_1A6, B029_1A7, B029_1G7, B029_1H1, B029_2D4, and B029_2H1, and antigen binding fragments thereof, such as antigen binding fragments that comprise the CDRs of these lead antibody clones. The amino acid sequences of these antibodies, and their CDR regions, are provided in the below tables, which also provides SEQ ID NOs for each amino acid sequence.

TABLE 1
B029_1A6 Anti-PRAME Antibody Heavy & Light Chain Sequences

| Clone/Region | SEQ ID NO. | Amino Acid or Nucleotide Sequence |
|---|---|---|
| Light chain | SEQ ID NO. 1 | EIVLTQSPGTLSLSPGERATLSCRASQSVSSNSLAWYQQKPGQAPRLLIYDASSRATGI |
| variable domain | | PDRFSGSGSGTDFTLTISRLEPEDFAVYYCQQYESSPLTFGQGTKVEIK |
| aa | | (amino acids shown in bold & underlined are the 3 CDRs) |
| Light chain | SEQ ID NO. 2 | GAGATTGTGCTGACACAGAGCCCCGGCACACTGTCACTTTCTCCAGGCGAAAGAGCC |
| variable domain | | ACACTGAGCTGCAGAGCCAGCCAGAGCGTGTCCTCTAATAGCCTGGCCTGGTATCAG |
| Nt | | CAGAAGCCCGGACAAGCTCCCCGGCTGCTGATCTACGATGCCTCTTCTAGAGCCACCG |
| | | GCATTCCCGACAGATTTTCTGGCAGCGGCTCCGGCACCGATTTCACCCTGACAATCAG |
| | | CAGACTGGAACCCGAGGACTTCGCCGTGTACTACTGCCAGCAGTACGAGAGCAGCCC |
| | | TCTGACATTTGGCCAGGGCACCAAGGTGGAAATCAAG |
| Heavy chain | SEQ ID NO. 3 | EVQLLESGGGLVQPGGSLRLSCAASGFTESNYAMSWVRQAPGKGLEWVSAISGSGGS |
| variable domain | | TYYADSVKGRFTISRDNSKNTLYLQMNSLRAEDTAVYYCARGGLVLSPWGQGTLVTV |
| aa | | SS |
| | | (amino acids shown in bold & underlined are the 3 CDRs) |
| Heavy chain | SEQ ID NO. 4 | GAAGTTCAGCTGCTGGAATCTGGCGGCGGACTGGTTCAACCTGGCGGATC |
| variable domain | | TCTGAGACTGAGCTGTGCCGCCAGCGGCTTCACCTTCAGCAATTACGCCA |
| nt | | TGAGCTGGGTCCGACAGGCCCCTGGAAAAGGCCTTGAATGGGTGTCCGCC |
| | | ATCTCTGGCAGCGGCGGCAGCACATATTACGCCGATTCTGTGAAGGGCAG |
| | | ATTCACCATCAGCCGGGACAACAGCAAGAACACCCTGTACCTGCAGATG |
| | | AACAGCCTGAGAGCCGAGGACACCGCCGTGTACTATTGTGCTAGAGGTG |
| | | GCCTGGTGCTGAGCCCTTGGGGACAGGGAACACTGGTCACAGTGTCTAGC |
| Light chain | SEQ ID NO. 5 | QSVSSNS |
| CDRL1 | | |
| Light chain | SEQ ID NO. 6 | DAS |
| CDRL2 | | |
| Light chain | SEQ ID NO. 7 | QQYESSPLT |
| CDRL 3 | | |
| Heavy chain | SEQ ID NO. 8 | GFTFSNYA |
| CDRL1 | | |
| Heavy chain | SEQ ID NO. 9 | ISGSGGST |
| CDRL2 | | |
| Heavy chain | SEQ ID NO. 10 | ARGGLVLSP |
| CDRL 3 | | |

TABLE 2
B029_1A7 Anti-PRAME Antibody Heavy & Light Chain Sequences

| Clone/Region | SEQ ID NO. | Amino Acid or Nucleotide Sequence |
|---|---|---|
| Light chain | SEQ ID NO. 11 | DIVMTQSPDSLAVSLGERATINCKSSQSVLYSYNNKNRLAWYQQKPGQPPK |
| variable domain | | LLIYDASTRESGVPDRFSGSGSGTDFTLTISSLQAEDVAVYYCQQSYSEPITFG |
| aa | | QGTKVEIK |
| | | (amino acids shown in bold & underlined are the 3 CDRs) |
| Light chain | SEQ ID NO. 12 | GATATCGTGATGACACAGAGCCCCGATAGCCTGGCCGTGTCTCTGGGAGA |
| variable domain | | AAGAGCCACCATCAACTGCAAGAGCAGCCAGAGCGTGCTGTACTCCTAC |
| Nt | | AACAACAAGAACCGGCTGGCCTGGTATCAGCAGAAGCCTGGACAGCCTC |
| | | CTAAGCTGCTGATCTACGATGCCAGCACCAGAGAAAGCGGCGTGCCCGAT |
| | | AGATTTTCTGGCAGCGGCTCTGGCACCGACTTCACCCTGACAATTAGCTC |
| | | CCTGCAGGCCGAGGATGTGGCCGTGTACTACTGT |
| | | CAGCAGAGCTACAGCGAGCCCATCACCTTTGGCCAGGGCACCAAGGTGG |
| | | AAATCAAG |
| Heavy chain | SEQ ID NO. 13 | EVQLLESGGGLVQPGGSLRLSCAASGFTFSSYWMSWVRQAPGKGLEWVSDI |
| variable domain | | SGSGGSTYYADSVKGRFTISRDNSKNTLYLQMNSLRAEDTAVYYCASLPDS |
| aa | | SGYYHWFDPWGQGTLVTVSS |
| | | (amino acids shown in bold & underlined are the 3 CDRs) |
| Heavy chain | SEQ ID NO. 14 | GAAGTTCAGCTGCTGGAATCTGGCGGCGGACTGGTTCAACCTGGCGGATC |
| variable domain | | TCTGAGACTGAGCTGTGCCGCCAGCGGCTTCACCTTTAGCAGCTACTGGA |
| nt | | TGAGCTGGGTCCGACAGGCCCCTGGCAAAGGACTTGAATGGGTGTCCGAT |
| | | ATCAGCGGCTCTGGCGGCAGCACCTACTACGCCGATTCTGTGAAGGGCAG |
| | | ATTCACCATCAGCCGGGACAACAGCAAGAACACCCTGTACCTGCAGATG |
| | | AACAGCCTGAGAGCCGAGGACACCGCCGTGTACTATTGTGCCAGCCTGCC |
| | | TGATAGCAGCGGCTACTACCATTGGTTCGACCCTTGGGGCCAGGGCACAC |
| | | TGGTTACAGTGTCTAGC |
| Light chain | SEQ ID NO. 15 | QSVLYSYNNKNR |
| CDRL1 | | |
| Light chain | SEQ ID NO. 16 | DAS |
| CDRL2 | | |
| Light chain | SEQ ID NO. 17 | QQSYSEPIT |
| CDRL3 | | |
| Heavy chain | SEQ ID NO. 18 | GFTFSSYW |
| CDRL1 | | |
| Heavy chain | SEQ ID NO. 19 | ISGSGGST |
| CDRL2 | | |
| Heavy chain | SEQ ID NO. 20 | ASLPDSSGYYHWFDP |
| CDRL3 | | |

TABLE 3
B029_1G7 Anti-PRAME Antibody Heavy & Light Chain Sequences

| Clone/Region | SEQ ID NO. | Amino Acid or Nucleotide Sequence |
|---|---|---|
| Light chain | SEQ ID NO. 21 | DIVMTQSPDSLAVSLGERATINCKSSQSVLYSSNNENYLAWYQQKPGQPPKL |
| variable domain | | LIYAASTRESGVPDRFSGSGSGTDFTLTISSLQAEDVAVYYCQQWYSAPYTF |
| aa | | GQGTKVEIK |
| Light chain | SEQ ID NO. 22 | GATATCGTGATGACACAGAGCCCCGATAGCCTGGCCGTGTCTCTGGGAGA |
| variable domain | | AAGAGCCACCATCAACTGCAAGAGCAGCCAGAGCGTGCTGTACTCCAGC |
| Nt | | AACAACGAGAACTACCTGGCCTGGTATCAGCAGAAGCCTGGCCAGCCTCC |
| | | TAAGCTGCTGATCTACGCTGCCAGCACCAGAGAAAGCGGCGTGCCCGATA |
| | | GATTTTCTGGCAGCGGCTCTGGCACCGACTTCACCCTGACAATTAGCTCCC |
| | | TGCAGGCCGAGGATGTGGCCGTGTACTATTGC |
| | | CAGCAGTGGTACAGCGCCCCTTACACCTTTGGCCAGGGCACCAAGGTGGA |
| | | AATCAAG |
| Heavy chain | SEQ ID NO. 23 | EVQLLESGGGLVQPGGSLRLSCAASGFTFTDSAMSWVRQAPGKGLEWVSDI |
| variable domain | | DGSGSGGGTYYADSVKGRFTISRDNSKNTLYLQMNSLRAEDTAVYYCATD |
| aa | | RGWGTFDFWGQGTLVTVSS |
| Heavy chain | SEQ ID NO. 24 | GAAGTTCAGCTGCTGGAATCTGGCGGCGGACTGGTTCAACCTGGCGGATC |
| variable domain | | TCTGAGACTGAGCTGTGCCGCCAGCGGCTTCACCTTTACCGATAGCGCCA |
| nt | | TGAGCTGGGTCCGACAGGCTCCTGGAAAAGGCCTGGAATGGGTGTCCGA |
| | | CATCGATGGCAGTGGATCTGGCGGAGGCACCTACTACGCCGATTCTGTGA |
| | | AGGGCAGATTCACCATCAGCCGGGACAACAGCAAGAACACCCTGTACCT |
| | | GCAGATGAACAGCCTGAGAGCCGAGGACACCGCCGTGTACTACTGTGCC |
| | | ACAGATAGAGGCTGGGGCACCTTCGATTTTTGGGGCCAGGGAACCCTGGT |
| | | CACCGTGTCTAGC |
| Light chain | SEQ ID NO. 25 | QSVLYSSNNENY |
| CDRL1 | | |
| Light chain | SEQ ID NO. 26 | AAS |
| CDRL2 | | |
| Light chain | SEQ ED NO. 27 | QQWYSAPYT |
| CDRL3 | | |
| Heavy chain | SEQ ID NO. 28 | GFTFTDSA |
| CDRL1 | | |
| Heavy chain | SEQ ID NO. 29 | IDGSGSGGGT |
| CDRL2 | | |
| Heavy chain | SEQ ID NO. 30 | ATDRGWGTFDF |
| CDRL3 | | |

TABLE 4
B029_1H1 Anti-PRAME Antibody Heavy & Light Chain Sequences

| Clone/Region | SEQ ID NO. | Amino Acid or Nucleotide Sequence |
|---|---|---|
| Light chain | SEQ ID NO. 31 | DIVMTQSPDSLAVSLGERATINCKSSQSVLYSGNNKNYLAWYQQKPGQPPKLLIYAAS |
| variable domain | | TRESGVPDRFSG SGSGTDFTLTISSLQAEDVAVYYCQQYDERPITFGQGTKVEIK |
| aa | | |
| Light chain | SEQ ID NO. 32 | GATATCGTGATGACACAGAGCCCCGATAGCCTGGCCGTGTCTCTGGGAGAAAGAGCC |
| variable domain | | ACCATCAACTGCAAGAGCAGCCAGAGCGTGCTGTACTCCGGCAACAACAAGAACTAC |
| Nt | | CTGGCCTGGTATCAGCAGAAGCCCGGCCAGCCTCCTAAGCTGCTGATCTATGCTGCCA |
| | | GCACCAGAGAAAGCGGCGTGCCCGATAGATTTTCTGGCAGCGGCTCTGGCACCGACT |
| | | TCACCCTGACAATTAGCTCCCTGCAGGCCGAGGATGTGGCCGTGTACTACTGC |
| | | CAGCAGTACGACGAGAGGCCCATCACATTTGGCCAGGGCACCAAGGTGGAAATCAAG |
| Heavy chain | SEQ ID NO. 33 | EVQLLESGGGLVQPGGSLRLSCAASGFTFSSYAMSWVRQAPGKGLEWVSEIDGEGDS |
| variable domain | | TKYADSVKGRFTI |
| aa | | SRDNSKNTLYLQMNSLRAEDTAVYYCAKEYYDIFDGTDVVVGQGTTVTVSS |
| Heavy chain | SEQ ID NO. 34 | GAAGTTCAGCTGCTGGAATCTGGCGGCGGACTGGTTCAACCTGGCGGATCTCTGAGA |
| variable domain | | CTGAGCTGTGCCGCCAGCGGCTTCACCTTTAGCAGCTACGCCATGAGCTGGGTCCGAC |
| nt | | AGGCTCCTGGCAAAGGCCTTGAATGGGTGTCCGAGATTGACGGCGAGGGCGACAGCA |
| | | CCAAATACGCCGATTCTGTGAAGGGCAGATTCACCATCAGCCGGGACAACAGCAAGA |
| | | ACACCCTGTACCTGCAGATGAACAGCCTGAGAGCCGAGGACACCGCCGTGTACTACT |
| | | GCGCCAAAGAGTACTACGACATCTTCGACGGCACCGACGTGTGGGGCCAGGGAACAA |
| | | CAGTGACAGTGTCTAGC |
| Light chain | SEQ ID NO. 35 | QSVLYSGNNKNY |
| CDRL1 | | |
| Light chain | SEQ ID NO. 36 | AAS |
| CDRL2 | | |
| Light chain | SEQ ID NO. 37 | QQYDERPIT |
| CDRL 3 | | |
| Heavy chain | SEQ ID NO. 38 | GFTFSSYA |
| CDRL1 | | |
| Heavy chain | SEQ ID NO. 39 | IDGEGDST |
| CDRL2 | | |
| Heavy chain | SEQ ID NO. 40 | AKEYYDIFDGTDV |
| CDRL 3 | | |

TABLE 5
B029_2D4 Anti-PRAME Antibody Heavy & Light Chain Sequences

| Clone/Region | SEQ ID NO. | Amino Acid or Nucleotide Sequence |
|---|---|---|
| Light chain | SEQ ID NO. 41 | EIVLTQSPGTLSLSPGERATLSCRASQSVSSSYLAWYQQKPGQAPRLLIYGASSRATGIP |
| variable domain | | DRFSGSGSGT DFTLTISRLEPEDFAVYYCQQYESAPLTFGQGTKVEIK |
| aa | | |
| Light chain | SEQ ID NO. 42 | GAGATTGTGCTGACACAGAGCCCCGGCACACTGTCACTTTCTCCAGGCGAAAGAGCC |
| variable domain | | ACACTGAGCTGCAGAGCCAGCCAGTCTGTGTCCAGCTCTTACCTGGCCTGGTATCAG |
| Nt | | CAGAAGCCTGGACAGGCTCCCCGGCTGTTGATCTATGGCGCCTCTTCTAGAGCCACCG |
| | | GCATTCCCGATAGATTCAGCGGCTCTGGCAGCGGCACCGATTTCACCCTGACAATCA |
| | | GCAGACTGGAACCCGAGGACTTCGCCGTGTACTACTGCCAGCAGTACGAGAGC |
| | | GCCCCTCTGACATTTGGCCAGGGCACCAAGGTGGAAATCAAG |
| Heavy chain | SEQ ID NO. 43 | EVQLLESGGGLVQPGGSLRLSCAASGFTESSYAMSWVRQAPGKGLEWVSAISGSGDS |
| variable domain | | TYYADSVKGRFTI |
| aa | | SRDNSKNTLYLQMNSLRAEDTAVYYCARDVDSFEGGMDVWGQGTTVTVSS |
| Heavy chain | SEQ ID NO. 44 | GAAGTTCAGCTGCTGGAATCTGGCGGCGGACTGGTTCAACCTGGCGGATCTCTGAGA |
| variable domain | | CTGAGCTGTGCCGCCAGCGGCTTCACCTTTAGCAGCTACGCCATGAGCTGGGTCCGAC |
| nt | | AGGCTCCTGGCAAAGGCCTTGAATGGGTGTCCGCCATCTCTGGCTCTGGCGACAGCA |
| | | CCTACTACGCCGATTCTGTGAAGGGCAGATTCACCATCAGCCGGGACAACAGCAAGA |
| | | ACACCCTGTACCTGCAGATGAACAGCCTGAGAGCCGAGGACACCGCCGTGTACTACT |
| | | GCGCTAGAGATGTGGACAGCTTCGAAGGCGGCATGGATGTGTGGGGCCAGGGAACAA |
| | | CAGTGACCGTGTCTAGC |
| Light chain | SEQ ID NO. 45 | QSVSSSY |
| CDRL1 | | |
| Light chain | SEQ ID NO. 46 | GAS |
| CDRL2 | | |
| Light chain | SEQ ID NO. 47 | QQYESAPLT |
| CDRL3 | | |
| Heavy chain | SEQ ID NO. 48 | GFTFSSYA |
| CDRL1 | | |
| Heavy chain | SEQ ID NO. 49 | ISGSGDST |
| CDRL2 | | |
| Heavy chain | SEQ ID NO. 50 | ARDVDSPEGGMDV |
| CDRL3 | | |

TABLE 6
B029_2H1 Anti-PRAME Antibody Heavy & Light Chain Sequences

| Clone/Region | SEQ ID NO. | Amino Acid or Nucleotide Sequence |
|---|---|---|
| Light chain | SEQ ID NO. 51 | EIVLTQSPGTLSLSPGERATLSCRASQSVSSTYLAWYQQKPGQAPRLLIYGAS |
| variable domain | | SRATGIPDRFSGSGSGTDFTLTISRLEPEDFAVYYCQQYSSAPFTFGQGTKVEI |
| aa | | K |
| Light chain | SEQ ID NO. 52 | GAGATTGTGCTGACACAGAGCCCCGGCACACTGTCACTTTCTCCAGGCGA |
| variable domain | | AAGAGCCACACTGAGCTGCAGAGCCAGCCAGTCCGTGTCTAGCACATACC |
| Nt | | TGGCCTGGTATCAGCAGAAGCCTGGACAGGCTCCCCGGCTGTTGATCTAT |
| | | GGCGCCTCTTCTAGAGCCACCGGCATTCCCGATAGATTCAGCGGCTCTGG |
| | | CAGCGGCACCGATTTCACCCTGACAATCAGCAGACTGGAACCCGAGGACT |
| | | TCGCCGTGTACTACTGCCAGCAGTACAGCAGCGCCCCTTTCACATTTGGC |
| | | CAGGGCACCAAGGTGGAAATCAAG |
| Heavy chain | SEQ ID NO. 53 | EVQLLESGGGLVQPGGSLRLSCAASGFTFTDYAMSWVRQAPGKGLEWVSW |
| variable domain | | ISGSGGSTKYADSVKGRFTISRDNSKNTLYLQMNSLRAEDTAVYYCAKCYD |
| aa | | ILTGYSIDYGMDVWGQGTTVTVSS |
| Heavy chain | SEQ ID NO. 54 | GAAGTTCAGCTGCTGGAATCTGGCGGCGGACTGGTTCAACCTGGCGGATC |
| variable domain | | TCTGAGACTGAGCTGTGCCGCCAGCGGCTTCACCTTTACCGATTACGCCA |
| nt | | TGAGCTGGGTCCGACAGGCCCCTGGAAAAGGCCTTGAATGGGTGTCCTGG |
| | | ATCTCTGGCTCTGGCGGCAGCACCAAATACGCCGATTCTGTGAAGGGCAG |
| | | ATTCACCATCAGCCGGGACAACAGCAAGAACACCCTGTACCTGCAGATG |
| | | AACAGCCTGAGAGCCGAGGACACCGCCGTGTACTACTGCGCCAAGTGCT |
| | | ACGATATCCTGACCGGCTACAGCATCGACTACGGCATGGATGTGTGGGGC |
| | | CAGGGCACAACCGTGACAGTGTCTAGC |
| Light chain | SEQ ID NO. 55 | QSVSSTY |
| CDRL1 | | |
| Light chain | SEQ ID NO. 56 | GAS |
| CDRL2 | | |
| Light chain | SEQ ID NO. 57 | QQYSSAPFT |
| CDRL 3 | | |
| Heavy chain | SEQ ID NO. 58 | GFTFTDYA |
| CDRL1 | | |
| Heavy chain | SEQ ID NO. 59 | ISGSGGST |
| CDRL2 | | |
| Heavy chain | SEQ ID NO. 60 | AKCYDILTGYSEDYGMDV |
| CDRL 3 | | |

TABLE 7
Sequence Identifier Summary (SEQ ID NO. Summary)

| | B029_1A6 | B029_1A7 | B029_1G7 | B029_1H1 | B029_2D4 | B029_2H1 |
|---|---|---|---|---|---|---|
| Light chain | SEQ ID NO. 1 | SEQ ID NO. 11 | SEQ ID NO. 21 | SEQ ID NO. 31 | SEQ ID NO. 41 | SEQ ID NO. 51 |
| variable | | | | | | |
| domain aa | | | | | | |
| Light chain | SEQ ID NO. 2 | SEQ ID NO. 12 | SEQ ID NO. 22 | SEQ ID NO. 32 | SEQ ID NO. 42 | SEQ ID NO. 52 |
| variable | | | | | | |
| domain | | | | | | |
| Nt | | | | | | |
| Heavy chain | SEQ ID NO. 3 | SEQ ID NO. 13 | SEQ ID NO. 23 | SEQ ID NO. 33 | SEQ ID NO. 43 | SEQ ID NO. 53 |
| variable | | | | | | |
| domain aa | | | | | | |
| Heavy chain | SEQ ID NO. 4 | SEQ ID NO. 14 | SEQ ID NO. 24 | SEQ ID NO. 34 | SEQ ID NO. 44 | SEQ ID NO. 54 |
| variable | | | | | | |
| domain | | | | | | |
| nt | | | | | | |
| Light chain | SEQ ID NO. 5 | SEQ ID NO. 15 | SEQ ID NO. 25 | SEQ ID NO. 35 | SEQ ID NO. 45 | SEQ ID NO. 55 |
| CDRL1 | | | | | | |
| Light chain | SEQ ID NO. 6 | SEQ ID NO. 16 | SEQ ID NO. 26 | SEQ ID NO. 36 | SEQ ID NO. 46 | SEQ ID NO. 56 |
| CDRL2 | | | | | | |
| Light chain | SEQ ID NO. 7 | SEQ ID NO. 17 | SEQ ID NO. 27 | SEQ ID NO. 37 | SEQ ID NO. 47 | SEQ ID NO. 57 |
| CDRL3 | | | | | | |
| Heavy chain | SEQ ID NO. 8 | SEQ ID NO. 18 | SEQ ID NO. 28 | SEQ ID NO. 38 | SEQ ID NO. 48 | SEQ ID NO. 58 |
| CDRL1 | | | | | | |
| Heavy chain | SEQ ID NO. 9 | SEQ ID NO. 19 | SEQ ID NO. 29 | SEQ ID NO. 39 | SEQ ID NO. 49 | SEQ ID NO. 59 |
| CDRL2 | | | | | | |
| Heavy chain | SEQ ID NO. 10 | SEQ ID NO. 20 | SEQ ID NO. 30 | SEQ ID NO. 40 | SEQ ID NO. 50 | SEQ ID NO. 60 |
| CDRL3 | | | | | | |

The antibodies described herein were produced in an IgG format. However, one of skill in the art will recognize, as described elsewhere herein, that these sequences can be engineered to different immunoglobulin formats, and/or to produce antigen binding fragments, and/or otherwise engineered (for example by humanization), while retaining the key determinants for PRAME binding—i.e. the CDRs.

In addition to providing the specific PRAME antibodies, and fragments thereof, whose sequences are provided in Tables 1 and 2 above, the present invention also encompasses variants and equivalents of these PRAME antibodies and antibody fragments. For example, such variants include humanized, chimeric, optimized, germlined, and/or other versions of any of the anTl-PRAME antibodies, or fragments thereof, disclosed herein. Likewise, in some embodiments variants of the specific sequences disclosed herein that comprise one or more substitutions, additions, deletions, or other mutations may be used. A VH and/or VL amino acid sequence or portion thereof, including a CDR sequence, can be, e.g., 85%, 90%, 95%, 96%, 97%, 98% or 99% similar to a sequence set forth herein, and/or comprise 1, 2, 3, 4, 5 or more substitutions, e.g., conservative substitutions, relative to a sequence set forth herein, such as a sequence from B029_1A6, B029_1A7, B029_1G7, B029_1H1, B029_2D4, or B029_2H1. In some embodiments a PRAME binding molecule according to the present invention comprises a VH and/or VL amino acid sequence, or portion thereof, that is 85%, 90%, 95%, 96%, 97%, 98% or 99% similar to that present in one of the specific sequences provided herein sequence set forth herein, and/or comprise 1, 2, 3, 4, 5 or more substitutions, e.g., conservative substitutions, relative to that sequence, but comprises the specific CDR sequences found within such VH and/or VL domains—i.e. any mutations (such as substitutions, additions, deletions, etc.) are outside of the CDRs. Such PRAME binding molecules, i.e. having VH and VL regions with a certain percent similarity to a VH region or VL region, or having one or more substitutions, e.g., conservative substitutions, can be obtained by mutagenesis (e.g., site-directed or PCR-mediated mutagenesis) of nucleic acid molecules encoding VH and/or VL regions described herein, followed by testing of the encoded altered binding molecule for binding to PRAME, and optionally testing for retained function of PRAME for example using the functional assays described herein.

Subsequent sections of this patent disclosure provide further details regarding different variants of the specific PRAME binding molecules described herein that are within the scope of the present invention, and how to make and use such variants.

In some embodiments, the PRAME binding molecule is a murine antibody, a human antibody, a humanized antibody, a chimeric antibody, a monoclonal antibody, a polyclonal antibody, a recombinant antibody, a bi-specific antibody, a multispecific antibody, or any combination thereof. In some embodiments, PRAME binding molecules comprise a Fab, a Fab′, a F(ab′)₂, a Fd, a Fv, a scFv, a disulfide linked Fv, a V-NAR domain, an IgNar, an intrabody, an IgGΔCH2, a minibody, a F(ab′)₃, a tetrabody, a triabody, a diabody, a single-domain antibody, DVD-Ig, Fcab, mAb², a (scFv)₂, or a scFv-Fc.

A PRAME binding molecule provided herein can include, in addition to a VH and a VL, a heavy chain constant region or fragment thereof. In certain aspects the heavy chain constant region is a human heavy chain constant region, e.g., a human IgG constant region, e.g., a human IgG1 constant region.

In certain embodiments, binding molecules of the invention are produced to comprise an altered Fc region, in which one or more alterations have been made in the Fc region in order to change functional and/or pharmacokinetic properties of the binding molecule. Such alterations may result in altered effector function, reduced immunogenicity, and/or an increased serum half-life. The Fc region interacts with a number of ligands, including Fc receptors, the complement protein C1q, and other molecules, such as proteins A and G. These interactions are essential for a variety of effector functions and downstream signaling events including antibody dependent cell-mediated cytotoxicity (ADCC) and complement dependent cytotoxicity (CDC). Accordingly, in certain embodiments the PRAME binding molecules of the invention have reduced or ablated affinity for an Fc ligand responsible for facilitating effector function, compared to a PRAME binding molecule not comprising the modification in the Fc region. In particular embodiments, the PRAME binding molecule has no ADCC activity and/or no CDC activity. In certain aspects, the PRAME binding molecule does not bind to an Fc receptor and/or complement factors. In certain aspects, the PRAME binding molecule has no effector function. Selecting particular constant domains to optimize desired effector functions is within the ordinary skill in the art. In some embodiments, the binding molecule is of the IgG1 subtype, and optionally comprises the TM format (L234F/L235E/P331S), as disclosed above in the Definitions section.

In certain aspects, a heavy chain constant region or fragment thereof can include one or more amino acid substitutions relative to a wild-type IgG constant domain, wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In certain aspects the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. More specifically, the IgG constant domain can contain amino acid substitutions relative to a wild-type human IgG constant domain including as substitution of the amino acid at Kabat position 252 with Tyrosine (Y), a substitution of the amino acid at Kabat position 254 with Threonine (T), and a substitution of the amino acid at Kabat position 256 with Glutamic acid (E). In some embodiments, the binding molecule is of the IgG1 subtype, and optionally comprises the triple mutant YTE, as disclosed supra in the Definitions section.

A PRAME binding molecule provided herein can include a light chain constant region or fragment thereof. In certain aspects the light chain constant region is a kappa constant region or a lambda constant region, e.g., a human kappa constant region or a human lambda constant region.

In certain aspects, this disclosure provides PRAME binding molecules that can specifically bind to the same PRAME epitope as a binding molecule comprising the heavy chain variable region (VH) and light chain variable region (VL) of any one of clones B029_1A6, B029_1A7, B029_1G7, B029_1H1, B029_2D4, and B029_2H1. The term “epitope” refers to a target protein determinant capable of binding to a binding molecule of the invention. Epitopes usually consist of chemically active surface groupings of molecules such as amino acids or sugar side chains, and usually have specific three-dimensional structural characteristics, as well as specific charge characteristics. Conformational and non-conformational epitopes are distinguished in that the binding to the former but not the latter is lost in the presence of denaturing solvents. Such binding molecules can be identified based on their ability to cross-compete (e.g., to competitively inhibit the binding of, in a statistically significant manner) with binding molecules, such as B029_1A6, B029_1A7, B029_1G7, B029_1H1, B029_2D4, and B029_2H1 in PRAME binding or activity assays.

Accordingly, in one embodiment, the invention provides PRAME binding molecules that compete for binding to PRAME with another PRAME binding molecule of the invention, such as B029_1A6, B029_1A7, B029_1G7, B029_1H1, B029_2D4, and B029_2H1. The ability of a binding molecule to inhibit the binding of B029_1A6, B029_1A7, B029_1G7, B029_1H1, B029_2D4, or B029_2H1 demonstrates that the test binding molecule can compete with B029_1A6, B029_1A7, B029_1G7, B029_1H1, B029_2D4, or B029_2H1 for binding to PRAME, such a binding molecule can, according to non-limiting theory, bind to the same or a related (e.g., a structurally similar or spatially proximal) epitope on PRAME as the PRAME binding molecule with which it competes. In one embodiment, an anti-PRAME antibody or antigen-binding fragment thereof binds to the same epitope on PRAME as B029_1A6, B029_1A7, B029_1G7, B029_1H1, B029_2D4, or B029_2H1. The term “competes” indicates that a binding molecule competes unidirectionally for binding to PRAME with B029_1A6, B029_1A7, B029_1G7, B029_1H1, B029_2D4, or B029_2H1. The term “cross-competes” indicates that a binding molecule competes bidirectionally for binding to PRAME with B029_1A6, B029_1A7, B029_1G7, B029_1H1, B029_2D4, or B029_2H1.

PRAME binding molecules provided herein can have beneficial properties. For example, the binding molecule can inhibit, suppress, or block various PRAME-mediated activities, which can be measured by assays known in the art.

In certain aspects, the binding molecules provided herein can bind to PRAME with a binding affinity characterized by a dissociation constant (K_{D}) of about 100 pM to about 0.5 nM as measured by a Biacore™ assay or on a Kinetic Exclusion Assay (KinExA) 3000 platform or on an Octet® instrument.

In certain aspects, an anti-PRAME antibody or antigen-binding fragment thereof can specifically bind to PRAME, or an antigenic fragment thereof, with a dissociation constant or K_{D} of less than 10⁻⁶ M, or of less than 10⁻⁷M, or of less than 10⁻⁸ M, or of less than 10⁻⁹M, or of less than 10⁻¹⁰ M, or of less than 10⁻¹¹ M, of less than 10⁻¹²M, of less than 10⁻¹³M, of less than 10⁻¹⁴ M, or of less than 10⁻¹⁵ M as measured, e.g., by Biacore™ or KinExA® or Octet®.

In another embodiment, a PRAME binding molecule of the invention binds to PRAME, or an antigenic fragment thereof, with a K_{off} of less than 1×10⁻³ s⁻¹, or less than 2×10⁻³ s⁻¹. In other embodiments, a PRAME binding molecule binds to PRAME, or an antigenic fragment thereof, with a K_{off} of less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹ as measured, e.g., by Biacore™ or KinExA® or Octet®.

In another embodiment, a PRAME binding molecule of the invention binds to PRAME, or an antigenic fragment thereof, with an association rate constant or Kₒₙ rate of at least 10⁵ M⁻¹ s⁻¹ at least 5×10⁵ M⁻¹ s⁻¹ at least 10⁶ M⁻¹ s⁻¹, at least 5×10⁶ M⁻¹ s⁻¹ at least 10⁷ M⁻¹ s⁻¹, at least 5×10⁷ M⁻¹ s⁻¹, or at least 10⁸M⁻¹ s⁻¹, or at least 10⁹M⁻¹ s⁻¹ as measured, e.g., by Biacore™ or KinExA® or Octet®.

The disclosure further provides a PRAME binding molecule that is conjugated to a heterologous agent. In certain aspects, the agent can be an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG), or a combination of two or more of any said agents.

In certain aspects, the disclosure provides a composition, e.g., a pharmaceutical composition, comprising a PRAME binding molecule of the invention, optionally further comprising one or more carriers, diluents, excipients, or other additives.

### III. Preparation of PRAME Binding Molecules

Monoclonal anti-PRAME antibodies can be prepared using hybridoma methods, such as those described by Kohler and Milstein Nature 256:495 (1975). Using the hybridoma method, a mouse, hamster, or other appropriate host animal, is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol (PEG), to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. RIA or ELISA) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid.

PRAME binding molecules can also be made using recombinant DNA methods, for example, as described in U.S. Pat. No. 4,816,567. In some instances, the polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains or antigen-binding fragments thereof are then cloned into suitable expression vectors, which when transfected into host cells such as E. coli cells, simian COS cells, Chinese hamster ovary (CHO) cells, or myeloma cells that do not otherwise produce immunoglobulin protein, binding molecules are generated by the host cells. Also, recombinant PRAME binding molecules can be isolated from phage display libraries expressing CDRs of the desired species, as described by McCafferty et al. (Nature, 348:552-554 (1990)); Clackson et al. (Nature, 352:624-628 (1991)); and Marks et al. (J. Mol. Biol., 222:581-597 (1991)). Production and expression of nucleic acids comprising nucleotide sequences encoding PRAME binding molecules are discussed in more detail in the next section.

The polynucleotide(s) encoding a binding molecule can further be modified in a number of different manners using recombinant DNA technology to generate alternative binding molecules. In some embodiments, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In certain embodiments, the PRAME binding molecule is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., J. Immunol. 147 (1):86-95 (1991); and U.S. Pat. No. 5,750,373).

The PRAME binding molecule can be selected from a phage library, where the phage library expresses human antibodies, as described, for example, by Vaughan et al. (Nat. Biotechnol., 14:309-314 (1996)), Sheets et al. (Proc. Nat'l. Acad. Sci. U.S.A. 95:6157-6162 (1998)), Hoogenboom et al. (J. Mol. Biol. 227:381 (1991)), and Marks et al. (J. Mol. Biol. 222:581 (1991)). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and in Rothe et al., J. Mol. Biol. 375:1182-1200 (2007).

Affinity maturation strategies and chain shuffling strategies are known in the art and can be employed to generate high affinity human antibodies or antigen-binding fragments thereof. (See Marks et al., Bio/Technology 10:779-783 (1992)).

In some embodiments, a PRAME binding molecule can be a humanized antibody or antigen-binding fragment thereof. Methods for engineering, humanizing, or resurfacing non-human or human antibodies can also be used and are well known in the art. A humanized, resurfaced, or similarly engineered antibody can have one or more amino acid residues from a source that is non-human, e.g., mouse, rat, rabbit, non-human primate, or other mammal. These non-human amino acid residues are replaced by residues that are often referred to as “import” residues, which are typically taken from an “import” variable, constant, or other domain of a known human sequence. Such imported sequences can be used to reduce immunogenicity or reduce, enhance, or modify binding, affinity, on-rate, off-rate, avidity, specificity, half-life, or any other suitable characteristic, as known in the art. In general, the CDR residues are directly and most substantially involved in influencing PRAME binding. Accordingly, part or all of the non-human or human CDR sequences are maintained while the non-human sequences of the variable and constant regions can be replaced with human or other amino acids. Humanization, resurfacing, or engineering of PRAME antibodies or antigen-binding fragments thereof can be performed using any known method, such as, but not limited to, those described in, Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246.

Anti-PRAME humanized antibodies and antigen-binding fragments thereof can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments the PRAME binding molecule is anti-PRAME antibody fragment. Various techniques are known for the production of antibody fragments. Traditionally, these fragments are derived via proteolytic digestion of intact antibodies. See, e.g., Morimoto et al., J. Biochem. Biophys. Meth. 24:107-117 (1993); Brennan et al., Science, 229:81-83 (1985). In certain embodiments, anti-PRAME antibody fragments are produced recombinantly. Fab, Fv, and scFv antibody fragments can all be expressed in and secreted from E. coli or other host cells, thus allowing the production of large amounts of these fragments. Such anti-PRAME antibody fragments can also be isolated from the antibody phage libraries discussed above. Anti-PRAME antibody fragments can also be linear antibodies, as described in U.S. Pat. No. 5,641,870. Other techniques for the production of antibody fragments will be apparent to the skilled practitioner.

According to the present invention, techniques can be adapted for the production of single-chain antibodies specific to PRAME (see, e.g., U.S. Pat. No. 4,946,778). In addition, methods can be adapted for the construction of Fab expression libraries (see, e.g., Huse et al., Science 246:1275-1281 (1989)) to allow rapid and effective identification of monoclonal Fab fragments with the desired specificity for PRAME. Antibody fragments can also be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

In some aspects, the PRAME binding molecules can be modified in order to reduce or eliminate effector function. This can be achieved, for example, by the Triple Mutation™ L234F/L235E/P331S in the Fc domain of IgG1. Other mutations that reduce effector function are known in the art. See, e.g., Armour et al., Eur. J. Immunol. 29:2613-2624, 1999; Shields et al., J. Biol. Chem. 276:6591-6604, 2001.

In certain aspects, a PRAME binding molecule can be modified to increase its serum half-life. This can be achieved, for example, by incorporation of a salvage receptor binding epitope into the binding molecule by mutation of the appropriate region, or by incorporating the epitope into a peptide tag that is then fused to the binding molecule at either end or in the middle (e.g., by DNA or peptide synthesis), or by YTE mutation. Other methods to increase the serum half-life of an antibody or antigen-binding fragment thereof, e.g., conjugation to a heterologous molecule such as PEG, are known in the art.

Heteroconjugate PRAME antibodies and antigen-binding fragments thereof are also within the scope of the present invention. Heteroconjugate antibodies are composed of two covalently joined antibodies. Such antibodies have, for example, been proposed to target immune cells to unwanted cells (see, e.g., U.S. Pat. No. 4,676,980). It is contemplated that heteroconjugate anti-PRAME antibodies and antigen-binding fragments thereof can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

A PRAME binding molecule can be modified to contain additional chemical moieties not normally part of the protein. Such moieties can improve the characteristics of the binding molecule, for example, solubility, biological half-life, or absorption. The moieties can also reduce or eliminate any undesirable side effects of the binding molecule. An overview of those moieties can be found in Remington's Pharmaceutical Sciences, 20th ed., Mack Publishing Co., Easton, Pa. (2000).

### IV. Polynucleotides Encoding PRAME Binding Molecules, Preparation and Expression Thereof

This disclosure provides certain polynucleotides comprising nucleic acid sequences that encode PRAME binding molecules. The polynucleotides of the invention can be in the form of RNA or in the form of DNA. DNA includes cDNA, genomic DNA, and synthetic DNA; and can be double-stranded or single-stranded, and, if single stranded, can be the coding strand or non-coding (anti-sense) strand.

In certain embodiments, the polynucleotide can be isolated. In certain embodiments, the polynucleotide can be substantially pure. In certain embodiments, the polynucleotide can be cDNA or are derived from cDNA. In certain embodiments, the polynucleotide can be recombinantly produced. In certain embodiments, the polynucleotide can comprise the coding sequence for a mature polypeptide, fused in the same reading frame to a polynucleotide which aids, for example, in expression and optionally, secretion, of a polypeptide from a host cell (e.g., a promoter or other regulatory sequence, a leader sequence that functions as a secretory sequence for controlling transport of a polypeptide from the cell). The polypeptide having a leader sequence is a pre-protein and can have the leader sequence cleaved by the host cell to form the mature form of the polypeptide. The polynucleotide can also encode PRAME binding pro-protein which is the mature protein plus additional 5′ amino acid residues.

The disclosure provides an isolated polynucleotide comprising a nucleic acid encoding a PRAME binding molecule comprising an amino acid sequence from a VH and/or VL domain having 85%, 90%, 95%, 96%, 97%, 98% or 99% similarity to an amino acid sequence set forth herein, and/or comprising 1, 2, 3, 4, 5 or more amino acid substitutions, e.g., conservative substitutions, relative to an amino acid sequence set forth herein, such as a sequence from PRAME clones B029_1A6, B029_1A7, B029_1G7, B029_1H1, B029_2D4, or B029_2H1.

In certain embodiments the polynucleotide that comprises the coding sequence for the PRAME binding molecule is fused in the same reading frame as a marker sequence that allows, for example, for purification of the encoded polypeptide. For example, the marker sequence can be a hexa-histidine tag (SEQ ID NO. 61) supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

Polynucleotide variants are also provided. Polynucleotide variants can contain alterations in the coding regions, non-coding regions, or both. In some embodiments, polynucleotide variants contain alterations that produce silent substitutions, additions, or deletions, but do not alter the properties or activities of the encoded polypeptide. In some embodiments, polynucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

The invention includes vectors comprising the polynucleotides described above. Suitable vectors are described elsewhere herein, and are known to those of ordinary skill in the art. In some embodiments, a polynucleotide comprising a nucleic acid encoding a VH domain or portion thereof and the polynucleotide comprising a nucleic acid encoding a VL domain or portion thereof can reside in a single vector, or can be on separate vectors. Accordingly, the disclosure provides one or more vectors comprising the polynucleotides described above.

In certain aspects, the disclosure provides a composition, e.g., a pharmaceutical composition, comprising a polynucleotide or vector as described above, optionally further comprising one or more carriers, diluents, excipients, or other additives.

The disclosure further provides a host cell comprising a polynucleotide or vector of the invention, wherein the host cell can, in some instances, express a binding molecule that specifically binds to PRAME. Such a host cell can be utilized in a method of making a PRAME binding molecule, where the method includes (a) culturing the host cell and (b) isolating the binding molecule from the host cell or from the culture medium, if the binding molecule is secreted by the host cell.

In some embodiments a nucleotide sequence encoding a PRAME binding molecule can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a nucleotide oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed, e.g., by nucleotide sequencing, restriction mapping, and/or expression of a biologically active polypeptide in a suitable host. In order to obtain high expression levels of a transfected gene in a host, the gene can be operatively linked to or associated with transcriptional and translational expression control sequences that are functional in the chosen expression host.

In certain embodiments, recombinant expression vectors are used to amplify and express DNA encoding PRAME binding molecules. Recombinant expression vectors are replicable DNA constructs that have synthetic or cDNA-derived DNA fragments encoding a polypeptide chain of a PRAME binding molecule, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR 1, pBR322, pMB9 and their derivatives, wider host range plasmids, such as M13, and filamentous single-stranded DNA phages.

Suitable host cells for expression of a PRAME binding molecule include prokaryotes, yeast, insect, or higher eukaryotic cells under the control of appropriate promoters. Prokaryotes include gram negative or gram-positive organisms, for example E. coli or bacilli. Higher eukaryotic cells include established cell lines of mammalian origin as described below. Cell-free translation systems could also be employed. Additional information regarding methods of protein production, including antibody production, can be found in, e.g., U.S. Patent Publication No. 2008/0187954, U.S. Pat. Nos. 6,413,746 and 6,660,501, and International Patent Publication No. WO 04009823.

Various mammalian or insect cell culture systems can be advantageously employed to express recombinant PRAME binding molecules. Expression of recombinant proteins in mammalian cells can be performed because such proteins are generally correctly folded, appropriately modified, and completely functional. Examples of suitable mammalian host cell lines include 293 cells (e.g., HEK-293, HEK-293T, AD293), the COS-7 lines of monkey kidney cells described by Gluzman (Cell 23:175, (1981)), and other cell lines including, for example, L cells, C127, 3T3, Chinese hamster ovary (CHO), HeLa, and BHK cell lines. Mammalian expression vectors can comprise non-transcribed elements, such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are reviewed by Luckow and Summers (BioTechnology 6:47 (1988)).

PRAME binding molecules produced by a transformed host can be purified according to any suitable method. Such standard methods include chromatography (e.g., ion exchange, affinity, and sizing column chromatography), centrifugation, differential solubility, or by any other standard technique for protein purification. Affinity tags such as hexahistidine (SEQ ID NO. 61), maltose binding domain, influenza coat sequence, and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose, or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a PRAME binding molecule. Some or all of the foregoing purification steps, in various combinations, can also be employed to provide a homogeneous recombinant protein.

A recombinant PRAME binding molecule produced in bacterial culture can be isolated, for example, by initial extraction from cell pellets, followed by one or more concentration, salting-out, aqueous ion exchange, or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

### V. Use of PRAME Binding Molecules

The present invention provides various methods of using the PRAME binding molecules described herein. Such methods include, but are not limited to, use for inhibition of the proliferation of or killing tumor cells in vitro or in vivo, use in imaging for diagnostic purpose or for monitoring tumor progression, and the like.

This disclosure also provides for the use of a PRAME binding molecule as described herein in the manufacture of a medicament.

The PRAME binding molecules of the invention can be also be used for a variety of different applications, including those that involve detecting PRAME. Such methods may involve assaying the expression level PRAME for example by qualitatively or quantitatively measuring or estimating the level of PRAME in a first biological sample either directly (e.g., by determining or estimating absolute protein level) or relatively (e.g., by comparison to a second biological sample). For example, the PRAME expression level in a first biological sample can be measured or estimated and compared to a that of a standard or control taken from a second biological sample. A “biological sample” is a sample obtained from an individual, cell line, tissue culture, or other source of cells potentially expressing PRAME. Methods for obtaining tissue biopsies and body fluids from mammals are known in the art. The PRAME binding molecules of the invention can be used to assay PRAME protein levels in a biological sample using classical immunohistological methods known to those of skill in the art (e.g., see Jalkanen, et al., J. Cell. Biol. 101:976-985 (1985); Jalkanen et al., J. Cell Biol. 105:3087-3096 (1987)). Immunoassays that can be used include but are not limited to competitive and non-competitive assay systems using techniques such as Western blots, radioimmunoassays, ELISA (enzyme linked immunosorbent assay), ELISPOT, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and immunoelectron microscopy, to name some examples. Such assays are routine and well known in the art. Those skilled in the art will be able to determine operative and optimal assay conditions for each determination by employing routine experimentation.

Detection of PRAME can be facilitated by coupling the binding molecule to a detectable substance or label. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin, and aequorin. Examples of suitable radioactive material include 1251, 1311, 35S, or 3H.

In situ detection can be accomplished by removing a histological specimen, for example a tumor sample, from a subject, and contacting the specimen with a labeled PRAME binding molecule, or with a PRAME antibody and a labeled secondary antibody. Through the use of such a procedure, it is possible to determine not only the presence of PRAME, but also its distribution in the examined tissue. Using the present invention, those of ordinary skill will readily perceive that any of a wide variety of histological methods (such as staining procedures) can be modified in order to achieve such in situ detection.

### VI. Kits Comprising PRAME Binding Molecules

This disclosure further provides kits that comprise a PRAME binding molecule, which can be used to perform the methods described herein. In certain embodiments, a kit comprises at least one purified PRAME binding molecule in one or more containers. In some embodiments, the kit contains one or more of the components necessary and/or sufficient to perform a detection assay, including controls, directions for performing assays, and any necessary software for analysis and presentation of results. One skilled in the art will readily recognize that the disclosed PRAME binding molecules can be readily incorporated into any of the established kit formats that are well known in the art.

Embodiments of the present disclosure can be further described and understood by reference to the following non-limiting “Examples,” which describe in the preparation of certain exemplary PRAME binding molecules, some exemplary characterization of such molecules, and some exemplary methods for using such binding molecules. It will be apparent to those skilled in the art that many modifications to the specific description provided in the Examples can be practiced without undue experimentation and without departing from the scope of the present disclosure.

### Example

### Generation & Testing of PRAME Antibodies

A portion of the PRAME protein (UniProt accession number P78395) corresponding to amino acids Arg310-Asn331, which are predicted to be exposed on the extracellular side of the plasma membrane, was synthesized as a peptide conjugated to either biotin or bovine serum albumin (BSA) for in vitro antibody generation.

A proprietary naïve, semi-synthetic scFv phage display library was screened for antibodies that bind the PRAME peptide by using standard solution phage display panning techniques. PRAME peptide conjugated to biotin was incubated with the phage library and captured with paramagnetic streptavidin beads, followed by standard washing, elution and phage amplification steps. Prior to incubating the phage library with PRAME peptide, the library was depleted of non-specific binding phage by incubation with a PRAME family consensus sequence peptide conjugated to biotin to remove all phage that display antibodies that bind PRAME homologs. The entire process of panning was repeated 3 times, using amplified PRAME target binder-enriched phage pools from the previous round of panning as input for subsequent rounds.

In order to identify clones that showed high specificity for the PRAME peptide, single clones from the third round of panning were analyzed for binding to the PRAME peptide, the PRAME homolog consensus peptide, and BSA as a non-specific control by enzyme-linked immunosorbent assay (ELISA). Monoclonal phage supernatants were tested for binding to all three antigens and only those that showed PRAME-specific binding were selected for antibody sequencing. 184 PRAME binding phage clones yielded 35 membrane target hits with 6 unique sequences. Clones with unique antibody sequences were chosen for further analysis.

PRAME binding was confirmed b=by flow cytometry using concentrated monoclonal phage preparations and THP-1 (PRAME⁺, human monocytic leukemia) and KG-1 (PRAME⁻, acute myelogenous leukemia) cell lines. Phage was detected with an anti-M13 phage monoclonal antibody.

Six unique antibodies (B029_1A6, B029_1A7, B029_1G7, B029_1H1, B029_2D4, and B029_2H1) that showed specific binding to the PRAME peptide and to PRAME⁺ cells were reformatted to full-length human IgG1 molecules using standard molecular cloning techniques.

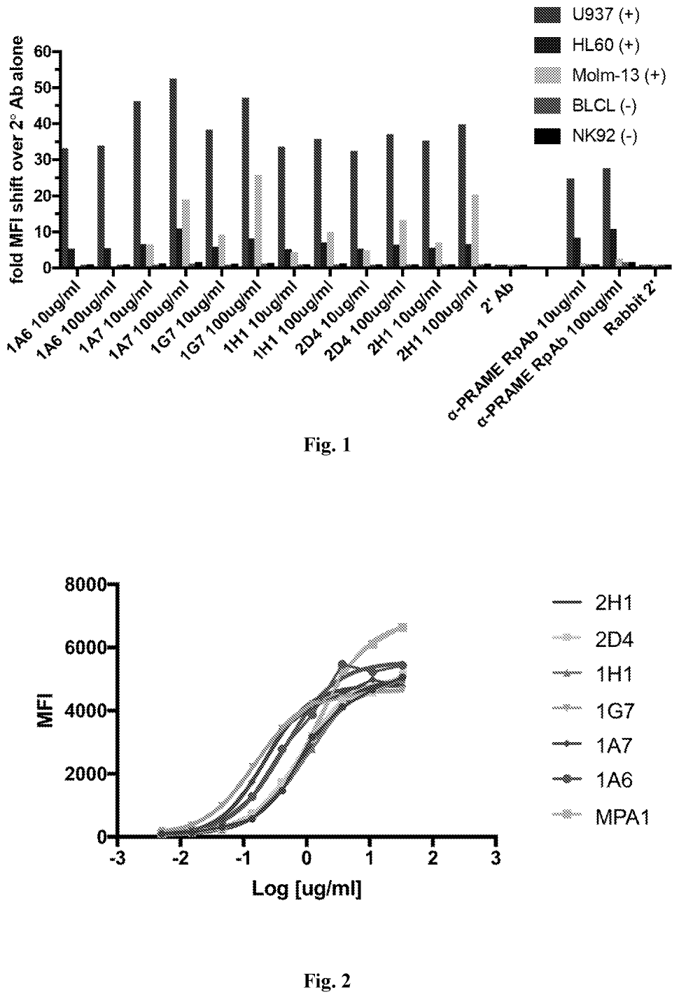

FIG. 1 shows flow cytometry analyses of mAb binding to 6 cell lines: U937, PRAME⁺ human myeloid leukemia; HL60, PRAME⁺ human acute promyelocytic leukemia; Molm-13 PRAME⁺ human acute monocytic leukemia; BLCL, PRAME⁻ Epstein-Barr virus-transformed lymphoblastoid B cells; and NK92, PRAME⁻ immortalized natural killer-like cells.

FIG. 2 shows an estimation of antibody affinity to THP-1 cells determined by flow cytometry with titrated concentrations of antibody, and EC₅₀ values for binding. MPA1 (Pankov et al., 2017), a rabbit anti-PRAME polyclonal antibody was included as reference for comparison. The EC₅₀ values were as shown in Table 8, below.

TABLE 8

| | EC₅₀ (mg/ml) | EC₅₀ (nM) |
|---|---|---|
| B029_2H1 | 0.879 | 5.86 |
| B029_2D4 | 0.795 | 5.30 |
| B029_1H1 | 0.974 | 6.49 |
| B029_1G7 | 0.157 | 1.05 |
| B029_1A7 | 0.218 | 1.45 |
| B029_1A6 | 0.448 | 2.99 |
| MPA1 | 1.39 | 9.27 |

Additional methods and protocols not specifically described in the text of Examples below can be found in “Phage Display: A Laboratory Manual”, (Barbas, 2001), the contents of which are hereby incorporated by reference.

1. An isolated PRAME binding molecule comprising:
(a) (i) a heavy chain variable region comprising: a CDR H1 domain comprising SEQ ID NO. 8, a CDR H2 domain comprising SEQ ID NO. 9, and a CDR H3 domain comprising SEQ ID NO. 10, and
(ii) a light chain variable region comprising: a CDR L1 domain comprising SEQ ID NO. 5, a CDR L2 domain comprising SEQ ID NO. 6, a CDR L3 domain comprising SEQ ID NO. 7; or
(b) (i) a heavy chain variable region comprising: a CDR H1 domain comprising SEQ ID NO. 18, a CDR H2 domain comprising SEQ ID NO. 19, and a CDR H3 domain comprising SEQ ID NO. 20, and
(ii) a light chain variable region comprising: a CDR L1 domain comprising SEQ ID NO. 15, a CDR L2 domain comprising SEQ ID NO. 16, a CDR L3 domain comprising SEQ ID NO. 17; or
(c) (i) a heavy chain variable region comprising: a CDR H1 domain comprising SEQ ID NO. 28, a CDR H2 domain comprising SEQ ID NO. 29, and a CDR H3 domain comprising SEQ ID NO. 30, and
(ii) a light chain variable region comprising: a CDR L1 domain comprising SEQ ID NO. 25, a CDR L2 domain comprising SEQ ID NO. 26, a CDR L3 domain comprising SEQ ID NO. 27; or
(d) (i) a heavy chain variable region comprising: a CDR H1 domain comprising SEQ ID NO. 38, a CDR H2 domain comprising SEQ ID NO. 39, and a CDR H3 domain comprising SEQ ID NO. 40, and
(ii) a light chain variable region comprising: a CDR L1 domain comprising SEQ ID NO. 35, a CDR L2 domain comprising SEQ ID NO. 36, a CDR L3 domain comprising SEQ ID NO. 37; or
(e) (i) a heavy chain variable region comprising: a CDR H1 domain comprising SEQ ID NO. 48, a CDR H2 domain comprising SEQ ID NO. 49, and a CDR H3 domain comprising SEQ ID NO. 50, and
(ii) a light chain variable region comprising: a CDR L1 domain comprising SEQ ID NO. 45, a CDR L2 domain comprising SEQ ID NO. 46, a CDR L3 domain comprising SEQ ID NO. 47; or
(f) (i) a heavy chain variable region comprising: a CDR H1 domain comprising SEQ ID NO. 58, a CDR H2 domain comprising SEQ ID NO. 59, and a CDR H3 domain comprising SEQ ID NO. 60, and
(ii) a light chain variable region comprising: a CDR L1 domain comprising SEQ ID NO. 55, a CDR L2 domain comprising SEQ ID NO. 56, a CDR L3 domain comprising SEQ ID NO. 57.

2. An isolated PRAME binding molecule comprising:
(a) (i) a heavy chain variable region comprising SEQ ID NO. 3, and
(ii) a light chain variable region comprising f SEQ ID NO.1; or
(b) (i) a heavy chain variable region comprising SEQ ID NO. 13, and
(ii) a light chain variable region comprising SEQ ID NO. 11; or
(c) (i) a heavy chain variable region comprising SEQ ID NO. 23, and
(ii) a light chain variable region comprising f SEQ ID NO.21; or
(d) (i) a heavy chain variable region comprising SEQ ID NO. 33, and
(ii) a light chain variable region comprising f SEQ ID NO.31; or
(e) (i) a heavy chain variable region comprising SEQ ID NO. 43, and
(ii) a light chain variable region comprising f SEQ ID NO.41; or
(f) (i) a heavy chain variable region comprising SEQ ID NO. 53, and
(ii) a light chain variable region comprising f SEQ ID NO.51.

3. An isolated PRAME binding molecule that specifically binds to the same epitope on PRAME as a PRAME binding molecule according to claim 1 or claim 2.

4. An isolated PRAME binding molecule that competes with a PRAME binding molecule according to claim 1 or claim 2 for binding to PRAME.

5. A PRAME binding molecule according to any of the preceding claims, wherein the binding molecule is an antibody.

6. A PRAME binding molecule according to claim 5, wherein the antibody is a humanized antibody, a fully human antibody, a murine antibody, a chimeric antibody, a monoclonal antibody, a polyclonal antibody, a bi-specific antibody, or a multi-specific antibody.

7. A PRAME binding molecule according to any of the preceding claims comprising a heavy chain constant region.

8. A PRAME binding molecule according to claim 7, wherein the heavy-chain constant region is selected from the group consisting of alpha, delta, epsilon, gamma, and mu heavy chain constant regions.

9. A PRAME binding molecule according to any of the preceding claims, comprising a light chain constant region.

10. A PRAME binding molecule according to claim 9, wherein the light chain constant region is a lambda light chain constant region or a kappa light chain constant region.

11. A PRAME binding molecule according to any of the preceding claims, wherein the binding molecule is an IgA, IgD, IgE, IgG or IgM class immunoglobulin.

12. A PRAME binding molecule according to any of claims 1-4, wherein the binding molecule is a Fv, a Fab, a F(ab′)2, a Fab′, a dsFv fragment, a single chain Fv (scFV), an sc(Fv)2, a disulfide-linked (dsFv), a diabody, a triabody, a tetrabody, a minibody, a, single chain antibody, a chimeric antigen receptor (CAR), or a bi-specific T cell engager (BiTE).

13. A PRAME binding molecule according to any of the preceding claims conjugated to a therapeutic agent or an imaging agent.

14. The PRAME binding molecule of claim 13, wherein the therapeutic agent is a chemotherapeutic agent of a radioactive agent.

15. The PRAME binding molecule of claim 13 or 14, wherein the imaging agent is a positron-emitting agent.

16. The PRAME binding molecule of claim 15, wherein the positron-emitting agent is zirconium-89.

17. A composition comprising a PRAME binding molecule according to any of the preceding claims.

18. A pharmaceutical composition comprising a PRAME binding molecule according to any of claims 1-16 and a pharmaceutically acceptable carrier.

19. A cell expressing a PRAME binding molecule according to any of claims 1-16.

20. The cell of claim 19, wherein the cell is a mammalian cell.

21. The cell of claim 19, wherein the cell is a human cell.

22. The cell of claim 19, wherein the cell is T cell and the PRAME binding molecule is a chimeric antigen receptor (CAR).

23. An isolated nucleic acid molecule comprising a nucleotide sequence encoding a PRAME binding molecule according to of any one of claims 1-16.

24. A vector comprising a nucleic acid molecule according to claim 23.

25. A cell comprising a nucleic acid molecule according to claim 23 or a vector according to claim 24.

26. The cell of claim 25, wherein the cell is a mammalian cell.

27. The cell of claim 25, wherein the cell is a human cell.

28. The cell of claim 25, wherein the cell is T cell and the PRAME binding molecule is a chimeric antigen receptor (CAR).

29. A method for inhibiting the proliferation of, or killing, tumor cells, the method comprising contacting tumor cells with an effective amount of a PRAME binding molecule according to any one of claims 1-16 or a composition according to claim 17 or claim 18 or a T cell according to claim 22 or claim 28.

30. The method of claim 29, wherein the tumor cells are selected from the group consisting of acute myeloid leukemia (AML), acute lymphoid leukemia (ALL), chronic myeloid leukemia (CML), ovarian carcinoma, endometrial carcinoma, lung carcinomas (e.g. squamous cell carcinoma of the lung), melanoma, (e.g. cutaneous melanoma), breast cancer (e.g. basal subtype breast cancer), medulloblastoma, neuroblastoma and osteosarcoma tumor cells.

31. The method of claim 29, wherein the tumor cells express PRAME.

32. A method for detecting PRAME in a tumor sample, the method comprising (a) contacting a tumor sample with a PRAME binding molecule according to any one of claims 1-16, or a composition according to claim 17 or claim 18, and (b) detecting binding of the PRAME binding molecule to PRAME, thereby detecting PRAME in the tumor sample.

33. The method of claim 32, wherein the tumor is selected from the group consisting of acute myeloid leukemia (AML), acute lymphoid leukemia (ALL), chronic myeloid leukemia (CML), ovarian carcinoma, endometrial carcinoma, lung carcinomas (e.g. squamous cell carcinoma of the lung), melanoma, (e.g. cutaneous melanoma), breast cancer (e.g. basal subtype breast cancer), medulloblastoma, neuroblastoma and osteosarcoma.


SEQUENCE LISTING (61 sequences)

SEQ ID NO: 1 - protein, 108 aa, Artificial Sequence
  Description of Artificial Sequence Synthetic polypeptide
EIVLTQSPGTLSLSPGERATLSCRASQSVSSNSLAWYQQKPGQAPRLLIYDASSRATGIPDRFSGSGSGTDFTLTISRLEPEDFAVYYCQQYESSPLTFGQGTKVEIK

SEQ ID NO: 2 - DNA, 324 bp, Artificial Sequence
  Description of Artificial Sequence Synthetic polynucleotide
gagattgtgctgacacagagccccggcacactgtcactttctccaggcgaaagagccacactgagctgcagagccagccagagcgtgtcctctaatagcctggcctggtatcagcagaagcccggacaagctccccggctgctgatctacgatgcctcttctagagccaccggcattcccgacagattttctggcagcggctccggcaccgatttcaccctgacaatcagcagactggaacccgaggacttcgccgtgtactactgccagcagtacgagagcagccctctgacatttggccagggcaccaaggtggaaatcaag

SEQ ID NO: 3 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCARGGLVLSPWGQGTLVTVSS

SEQ ID NO: 4 - DNA, 348 bp, Artificial Sequence
  Description of Artificial Sequence Synthetic polynucleotide
gaagttcagctgctggaatctggcggcggactggttcaacctggcggatctctgagactgagctgtgccgccagcggcttcaccttcagcaattacgccatgagctgggtccgacaggcccctggaaaaggccttgaatgggtgtccgccatctctggcagcggcggcagcacatattacgccgattctgtgaagggcagattcaccatcagccgggacaacagcaagaacaccctgtacctgcagatgaacagcctgagagccgaggacaccgccgtgtactattgtgctagaggtggcctggtgctgagcccttggggacagggaacactggtcacagtgtctagc

SEQ ID NO: 5 - protein, 7 aa, Artificial Sequence
  Description of Artificial Sequence Synthetic peptide
QSVSSNS

SEQ ID NO: 6 - protein, 3 aa, Artificial Sequence
  Description of Artificial Sequence Synthetic peptide
DAS

SEQ ID NO: 7 - protein, 9 aa, Artificial Sequence
  Description of Artificial Sequence Synthetic peptide
QQYESSPLT

SEQ ID NO: 8 - protein, 8 aa, Artificial Sequence
  Description of Artificial Sequence Synthetic peptide
GFTFSNYA

SEQ ID NO: 9 - protein, 8 aa, Artificial Sequence
  Description of Artificial Sequence Synthetic peptide
ISGSGGST

SEQ ID NO: 10 - protein, 9 aa, Artificial Sequence
  Description of Artificial Sequence Synthetic peptide
ARGGLVLSP

SEQ ID NO: 11 - protein, 113 aa, Artificial Sequence
  Description of Artificial Sequence Synthetic polypeptide
DIVMTQSPDSLAVSLGERATINCKSSQSVLYSYNNKNRLAWYQQKPGQPPKLLIYDASTRESGVPDRFSGSGSGTDFTLTISSLQAEDVAVYYCQQSYSEPITFGQGTKVEIK

SEQ ID NO: 12 - DNA, 339 bp, Artificial Sequence
  Description of Artificial Sequence Synthetic polynucleotide
gatatcgtgatgacacagagccccgatagcctggccgtgtctctgggagaaagagccaccatcaactgcaagagcagccagagcgtgctgtactcctacaacaacaagaaccggctggcctggtatcagcagaagcctggacagcctcctaagctgctgatctacgatgccagcaccagagaaagcggcgtgcccgatagattttctggcagcggctctggcaccgacttcaccctgacaattagctccctgcaggccgaggatgtggccgtgtactactgtcagcagagctacagcgagcccatcacctttggccagggcaccaaggtggaaatcaag

SEQ ID NO: 13 - protein, 122 aa, Artificial Sequence
  Description of Artificial Sequence Synthetic polypeptide
EVQLLESGGGLVQPGGSLRLSCAASGFTFSSYWMSWVRQAPGKGLEWVSDISGSGGSTYYADSVKGRFTISRDNSKNTLYLQMNSLRAEDTAVYYCASLPDSSGYYHWFDPWGQGTLVTVSS

SEQ ID NO: 14 - DNA, 366 bp, Artificial Sequence
  Description of Artificial Sequence Synthetic polynucleotide
gaagttcagctgctggaatctggcggcggactggttcaacctggcggatctctgagactgagctgtgccgccagcggcttcacctttagcagctactggatgagctgggtccgacaggcccctggcaaaggacttgaatgggtgtccgatatcagcggctctggcggcagcacctactacgccgattctgtgaagggcagattcaccatcagccgggacaacagcaagaacaccctgtacctgcagatgaacagcctgagagccgaggacaccgccgtgtactattgtgccagcctgcctgatagcagcggctactaccattggttcgacccttggggccagggcacactggttacagtgtctagc

SEQ ID NO: 15 - protein, 12 aa, Artificial Sequence
  Description of Artificial Sequence Synthetic peptide
QSVLYSYNNKNR

SEQ ID NO: 16 - protein, 3 aa, Artificial Sequence
  Description of Artificial Sequence Synthetic peptide
DAS

SEQ ID NO: 17 - protein, 9 aa, Artificial Sequence
  Description of Artificial Sequence Synthetic peptide
QQSYSEPIT

SEQ ID NO: 18 - protein, 8 aa, Artificial Sequence
  Description of Artificial Sequence Synthetic peptide
GFTFSSYW

SEQ ID NO: 19 - protein, 8 aa, Artificial Sequence
  Description of Artificial Sequence Synthetic peptide
ISGSGGST

SEQ ID NO: 20 - protein, 15 aa, Artificial Sequence
  Description of Artificial Sequence Synthetic peptide
ASLPDSSGYYHWFDP

SEQ ID NO: 21 - protein, 113 aa, Artificial Sequence
  Description of Artificial Sequence Synthetic polypeptide
DIVMTQSPDSLAVSLGERATINCKSSQSVLYSSNNENYLAWYQQKPGQPPKLLIYAASTRESGVPDRFSGSGSGTDFTLTISSLQAEDVAVYYCQQWYSAPYTFGQGTKVEIK

SEQ ID NO: 22 - DNA, 339 bp, Artificial Sequence
  Description of Artificial Sequence Synthetic polynucleotide
gatatcgtgatgacacagagccccgatagcctggccgtgtctctgggagaaagagccaccatcaactgcaagagcagccagagcgtgctgtactccagcaacaacgagaactacctggcctggtatcagcagaagcctggccagcctcctaagctgctgatctacgctgccagcaccagagaaagcggcgtgcccgatagattttctggcagcggctctggcaccgacttcaccctgacaattagctccctgcaggccgaggatgtggccgtgtactattgccagcagtggtacagcgccccttacacctttggccagggcaccaaggtggaaatcaag

SEQ ID NO: 23 - protein, 120 aa, Artificial Sequence
  Description of Artificial Sequence Synthetic polypeptide
EVQLLESGGGLVQPGGSLRLSCAASGFTFTDSAMSWVRQAPGKGLEWVSDIDGSGSGGGTYYADSVKGRFTISRDNSKNTLYLQMNSLRAEDTAVYYCATDRGWGTFDFWGQGTLVTVSS

SEQ ID NO: 24 - DNA, 360 bp, Artificial Sequence
  Description of Artificial Sequence Synthetic polynucleotide
gaagttcagctgctggaatctggcggcggactggttcaacctggcggatctctgagactgagctgtgccgccagcggcttcacctttaccgatagcgccatgagctgggtccgacaggctcctggaaaaggcctggaatgggtgtccgacatcgatggcagtggatctggcggaggcacctactacgccgattctgtgaagggcagattcaccatcagccgggacaacagcaagaacaccctgtacctgcagatgaacagcctgagagccgaggacaccgccgtgtactactgtgccacagatagaggctggggcaccttcgatttttggggccagggaaccctggtcaccgtgtctagc

SEQ ID NO: 25 - protein, 12 aa, Artificial Sequence
  Description of Artificial Sequence Synthetic peptide
QSVLYSSNNENY

SEQ ID NO: 26 - protein, 3 aa, Artificial Sequence
  Description of Artificial Sequence Synthetic peptide
AAS

SEQ ID NO: 27 - protein, 9 aa, Artificial Sequence
  Description of Artificial Sequence Synthetic peptide
QQWYSAPYT

SEQ ID NO: 28 - protein, 8 aa, Artificial Sequence
  Description of Artificial Sequence Synthetic peptide
GFTFTDSA

SEQ ID NO: 29 - protein, 10 aa, Artificial Sequence
  Description of Artificial Sequence Synthetic peptide
IDGSGSGGGT

SEQ ID NO: 30 - protein, 11 aa, Artificial Sequence
  Description of Artificial Sequence Synthetic peptide
ATDRGWGTFDF

SEQ ID NO: 31 - protein, 113 aa, Artificial Sequence
  Description of Artificial Sequence Synthetic polypeptide
DIVMTQSPDSLAVSLGERATINCKSSQSVLYSGNNKNYLAWYQQKPGQPPKLLIYAASTRESGVPDRFSGSGSGTDFTLTISSLQAEDVAVYYCQQYDERPITFGQGTKVEIK

SEQ ID NO: 32 - DNA, 339 bp, Artificial Sequence
  Description of Artificial Sequence Synthetic polynucleotide
gatatcgtgatgacacagagccccgatagcctggccgtgtctctgggagaaagagccaccatcaactgcaagagcagccagagcgtgctgtactccggcaacaacaagaactacctggcctggtatcagcagaagcccggccagcctcctaagctgctgatctatgctgccagcaccagagaaagcggcgtgcccgatagattttctggcagcggctctggcaccgacttcaccctgacaattagctccctgcaggccgaggatgtggccgtgtactactgccagcagtacgacgagaggcccatcacatttggccagggcaccaaggtggaaatcaag

SEQ ID NO: 33 - protein, 120 aa, Artificial Sequence
  Description of Artificial Sequence Synthetic polypeptide
EVQLLESGGGLVQPGGSLRLSCAASGFTFSSYAMSWVRQAPGKGLEWVSEIDGEGDSTKYADSVKGRFTISRDNSKNTLYLQMNSLRAEDTAVYYCAKEYYDIFDGTDVWGQGTTVTVSS

SEQ ID NO: 34 - DNA, 360 bp, Artificial Sequence
  Description of Artificial Sequence Synthetic polynucleotide
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgagattgacggcgagggcgacagcaccaaatacgccgattctgtgaagggcagattcaccatcagccgggacaacagcaagaacaccctgtacctgcagatgaacagcctgagagccgaggacaccgccgtgtactactgcgccaaagagtactacgacatcttcgacggcaccgacgtgtggggccagggaacaacagtgacagtgtctagc

SEQ ID NO: 35 - protein, 12 aa, Artificial Sequence
  Description of Artificial Sequence Synthetic peptide
QSVLYSGNNKNY

SEQ ID NO: 36 - protein, 3 aa, Artificial Sequence
  Description of Artificial Sequence Synthetic peptide
AAS

SEQ ID NO: 37 - protein, 9 aa, Artificial Sequence
  Description of Artificial Sequence Synthetic peptide
QQYDERPIT

SEQ ID NO: 38 - protein, 8 aa, Artificial Sequence
  Description of Artificial Sequence Synthetic peptide
GFTFSSYA

SEQ ID NO: 39 - protein, 8 aa, Artificial Sequence
  Description of Artificial Sequence Synthetic peptide
IDGEGDST

SEQ ID NO: 40 - protein, 13 aa, Artificial Sequence
  Description of Artificial Sequence Synthetic peptide
AKEYYDIFDGTDV

SEQ ID NO: 41 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYESAPLTFGQGTKVEIK

SEQ ID NO: 42 - DNA, 324 bp, Artificial Sequence
  Description of Artificial Sequence Synthetic polynucleotide
gagattgtgctgacacagagccccggcacactgtcactttctccaggcgaaagagccacactgagctgcagagccagccagtctgtgtccagctcttacctggcctggtatcagcagaagcctggacaggctccccggctgttgatctatggcgcctcttctagagccaccggcattcccgatagattcagcggctctggcagcggcaccgatttcaccctgacaatcagcagactggaacccgaggacttcgccgtgtactactgccagcagtacgagagcgcccctctgacatttggccagggcaccaaggtggaaatcaag

SEQ ID NO: 43 - protein, 120 aa, Artificial Sequence
  Description of Artificial Sequence Synthetic polypeptide
EVQLLESGGGLVQPGGSLRLSCAASGFTFSSYAMSWVRQAPGKGLEWVSAISGSGDSTYYADSVKGRFTISRDNSKNTLYLQMNSLRAEDTAVYYCARDVDSFEGGMDVWGQGTTVTVSS

SEQ ID NO: 44 - DNA, 360 bp, Artificial Sequence
  Description of Artificial Sequence Synthetic polynucleotide
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgccatctctggctctggcgacagcacctactacgccgattctgtgaagggcagattcaccatcagccgggacaacagcaagaacaccctgtacctgcagatgaacagcctgagagccgaggacaccgccgtgtactactgcgctagagatgtggacagcttcgaaggcggcatggatgtgtggggccagggaacaacagtgaccgtgtctagc

SEQ ID NO: 45 - protein, 7 aa, Artificial Sequence
  Description of Artificial Sequence Synthetic peptide
QSVSSSY

SEQ ID NO: 46 - protein, 3 aa, Artificial Sequence
  Description of Artificial Sequence Synthetic peptide
GAS

SEQ ID NO: 47 - protein, 9 aa, Artificial Sequence
  Description of Artificial Sequence Synthetic peptide
QQYESAPLT

SEQ ID NO: 48 - protein, 8 aa, Artificial Sequence
  Description of Artificial Sequence Synthetic peptide
GFTFSSYA

SEQ ID NO: 49 - protein, 8 aa, Artificial Sequence
  Description of Artificial Sequence Synthetic peptide
ISGSGDST

SEQ ID NO: 50 - protein, 13 aa, Artificial Sequence
  Description of Artificial Sequence Synthetic peptide
ARDVDSFEGGMDV

SEQ ID NO: 51 - protein, 108 aa, Artificial Sequence
  Description of Artificial Sequence Synthetic polypeptide
EIVLTQSPGTLSLSPGERATLSCRASQSVSSTYLAWYQQKPGQAPRLLIYGASSRATGIPDRFSGSGSGTDFTLTISRLEPEDFAVYYCQQYSSAPFTFGQGTKVEIK

SEQ ID NO: 52 - DNA, 324 bp, Artificial Sequence
  Description of Artificial Sequence Synthetic polynucleotide
gagattgtgctgacacagagccccggcacactgtcactttctccaggcgaaagagccacactgagctgcagagccagccagtccgtgtctagcacatacctggcctggtatcagcagaagcctggacaggctccccggctgttgatctatggcgcctcttctagagccaccggcattcccgatagattcagcggctctggcagcggcaccgatttcaccctgacaatcagcagactggaacccgaggacttcgccgtgtactactgccagcagtacagcagcgcccctttcacatttggccagggcaccaaggtggaaatcaag

SEQ ID NO: 53 - protein, 125 aa, Artificial Sequence
  Description of Artificial Sequence Synthetic polypeptide
EVQLLESGGGLVQPGGSLRLSCAASGFTFTDYAMSWVRQAPGKGLEWVSWISGSGGSTKYADSVKGRFTISRDNSKNTLYLQMNSLRAEDTAVYYCAKCYDILTGYSIDYGMDVWGQGTTVTVSS

SEQ ID NO: 54 - DNA, 375 bp, Artificial Sequence
  Description of Artificial Sequence Synthetic polynucleotide
gaagttcagctgctggaatctggcggcggactggttcaacctggcggatctctgagactgagctgtgccgccagcggcttcacctttaccgattacgccatgagctgggtccgacaggcccctggaaaaggccttgaatgggtgtcctggatctctggctctggcggcagcaccaaatacgccgattctgtgaagggcagattcaccatcagccgggacaacagcaagaacaccctgtacctgcagatgaacagcctgagagccgaggacaccgccgtgtactactgcgccaagtgctacgatatcctgaccggctacagcatcgactacggcatggatgtgtggggccagggcacaaccgtgacagtgtctagc

SEQ ID NO: 55 - protein, 7 aa, Artificial Sequence
  Description of Artificial Sequence Synthetic peptide
QSVSSTY

SEQ ID NO: 56 - protein, 3 aa, Artificial Sequence
  Description of Artificial Sequence Synthetic peptide
GAS

SEQ ID NO: 57 - protein, 9 aa, Artificial Sequence
  Description of Artificial Sequence Synthetic peptide
QQYSSAPFT

SEQ ID NO: 58 - protein, 8 aa, Artificial Sequence
  Description of Artificial Sequence Synthetic peptide
GFTFTDYA

SEQ ID NO: 59 - protein, 8 aa, Artificial Sequence
  Description of Artificial Sequence Synthetic peptide
ISGSGGST

SEQ ID NO: 60 - protein, 18 aa, Artificial Sequence
  Description of Artificial Sequence Synthetic peptide
AKCYDILTGYSIDYGMDV

SEQ ID NO: 61 - protein, 6 aa, Artificial Sequence
  Description of Artificial Sequence Synthetic 6xHis tag
HHHHHH